SEQUENCE LISTING (174 sequences)

SEQ ID NO: 1 - protein, 41 aa, Atrax robustus
GSQYCAPADQPCSLNTQPCCDDVTCTQERNENGHTAYYCRV

SEQ ID NO: 2 - protein, 98 aa, Hadronyche versuta
MKFSKLSLTLALILTQALFVLCGKINEDFMKNGLESQTLHDEIRKPIDSENPDTERLLDCLLDNRVCSSDRDCCGMTPSCTMGLCVPNVGGLVGGILG

SEQ ID NO: 3 - protein, 98 aa, Atrax robustus; identical to SEQ ID NO: 2

SEQ ID NO: 4 - protein, 98 aa, Hadronyche versuta
MKFSKLSLTLALILTQAIFVLCGKINEDFMKNDLESQALRDEIRKPIDSENPDTERLLDCLLDNRVCSSDKDCCGMTPSCTMGLCVPSVGGLVGGILG

SEQ ID NO: 5 - protein, 98 aa, Hadronyche versuta
MKFSKLSLTLALILTQAIFVLCGKINEDFMKNDLESHALHDEIRKPINSENPDTERLLDCLLDNRVCSSDKDCCGMTPSCTMGLCVPSVGGLVGGILG

SEQ ID NO: 6 - protein, 98 aa, Hadronyche versuta
MKFSKLSLTLALILTQALFVLCGKINEDFMKNGLESQALHDEIRKPIDSENPDTERLLDCLLDNRVCSSDKDCCGMTPSCTMGLCVPSVGGLVGGILG

SEQ ID NO: 7 - protein, 98 aa, Hadronyche versuta
MKFSKLSLTLALILTQVIFVLCGKINEDFMKNGLESQALHDEIRKPIDSENPDTERLLDCLLDNRVCSSDKDCCGMTPSCTMGLCVPSVGGLVGGILG

SEQ ID NO: 8 - protein, 93 aa, Hadronyche versuta
MKFSKLSLTLALILTQALFVLCDFMKNGLESQALHDEIRKSIDSENPDTERLLDCLLDNRVCSSDKDCCGMTPSCTMGLCVPSVGGLVGGILG

SEQ ID NO: 9 - protein, 98 aa, Atrax robustus
MKFSKLSLTLALILTQVLFVLCGKINEDFMKHGLESQALHDEIRKPIDSENPDTERLLDCLLDNRVCSSDKDCCGMTPSCTMGLCVPSVGGLVGGILG

SEQ ID NO: 10 - protein, 98 aa, Hadronyche versuta
MKFSKLSLTLALILTQAIFVLCGKINEDFMKNDLESQALHDEIRKPINSENPDTERLLDCLLDNRVCSSDKDCCGMTPSCTMGLCVPSVGGLVGGILG

SEQ ID NO: 11 - protein, 98 aa, Hadronyche versuta
MKFSKLSLTLALILTQALFVLCGKINEDFMKNGLESQALHDEIRKPIDSENPDTERLLDCLLDNRVCSSDRDCCGMTPSCTMGLCVPNVGGLVGDILG

SEQ ID NO: 12 - protein, 97 aa, Hadronyche versuta
MKFSKLSLTLALILTQVLFVLCGKIEDFMKNGLESQALHDEIRKPIDSENPDTERLLDCLLDNRVCSSDKDCCGMTPSCTMGLCVPNVGGLVGGILG

SEQ ID NO: 13 - protein, 93 aa, Hadronyche versuta
MKFSKLSLTLALILTQALFVLCDFMKNGLESQALHDEIRKPIDSENPDTERLLDCLLDNRVCSSDKDCCGMTPSCTMGLCVPNVGGLVGGILG

SEQ ID NO: 14 - protein, 98 aa, Hadronyche versuta
MKFSKLSLTLALILTQALFVLCGKINEDFMKNGLESQALHDEIRKPIDSENPDTERLLDCLLDNRICSSDKDCCGMTPSCTMGLCVPNVGGLVGGILG

SEQ ID NO: 15 - protein, 98 aa, Hadronyche versuta
MKFSKLSLTLALILTQALFVLCGKINEDFMKNGLESQALHDEIRKPIDSENPDTERLLDCLLDNRVCSSDKDCCGMTPSCTMGLCVPNVGGLVGGILG

SEQ ID NO: 16 - protein, 98 aa, Hadronyche versuta
MKFSKLSLTLALILTQAIFVLCGKINEDFMKNDLESQALHDEIRKPINSENPDTERLLDCLLDSRVCSSDKDCCGMTPSCTMGLCVPSVGGLVGGILG

SEQ ID NO: 17 - protein, 96 aa, Atrax robustus
MKFSKLSITLAVILTQAVFVFCGMTNEDFMEKGLESNELPDAIKKPVNSGKPDTKRLLDCVLSRMCFSNANCCGLTPPCKMGLCVPNVGGLLGGIL

SEQ ID NO: 18 - protein, 96 aa, Atrax robustus
MKFSKLSITLAVILTQAVFVFCGMTNEDFMEKGLESNELHDAIKKPVNSGKPDTERLLDCVLSRMCSSDANCCGLTPTCKMGLCVPNVGGLLGGIL

SEQ ID NO: 19 - protein, 101 aa, Hadronyche versuta
MKFSKLSLTLALILTQALFVLCGKINEDFMEHGLESHALHDEIRKPIDTEKADAERLVDCVVNTLGCSSDKDCCGMTPSCTLGICAPSVRGLVGGLLGRAL

SEQ ID NO: 20 - protein, 101 aa, Hadronyche versuta
MKFSKLSLTLALILTQALFVLCMKINEDFMENGLESHALHDEIRKPIDTEKADAERLVDCVVNTLGCSSDKDCCGMTPSCTLGICAPSVGGLVGGLLGRAL

SEQ ID NO: 21 - protein, 101 aa, Hadronyche versuta
MKFSKLSLTFALILTQALFVLCGKINEDFMDNGLESHALHDEIRKPIHTEKADAERLVDCVLNTLGCSSDKDCCGMTPSCTLGICAPSVGGLVGGLLGRAL

SEQ ID NO: 22 - protein, 101 aa, Hadronyche infensa
MKFSKLSLTLALILTQALFVLCGKINEDFMEHGLESHALHDEIRKPIDTEKADAERLVDCVVNTLGCSSDKDCCGMTPSCTLGICAPSVGGLVGGLLGRAL

SEQ ID NO: 23 - protein, 101 aa, Hadronyche infensa
MKFSKLSVTLALILTQTLLVLCGKINEDFMENGLESHALHDEIRKPIDTDKAYAERVLDCVVNTLGCSSDKDCCGMTPSCTLGICAPSVGGLVGGLLGRAL

SEQ ID NO: 24 - protein, 101 aa, Hadronyche versuta
MKFSKLSLTLALILTQALFVLCGKINEDFMENGLESHALHDEIRKPIDTEKADAERLVDCVVNTLGCSSDKDCCGMTPSCTLGICAPSVGGLVGGLLGRAL

SEQ ID NO: 25 - protein, 101 aa, Hadronyche versuta
MKFSKLSLTLALILTQALFVLCGKINEDFMENGLESHALHDEIRKPIDTEKADAERVLDCVVNTLGCSSDKDCCGMTPSCTLGICAPSVGGLVGGLLGRAL

SEQ ID NO: 26 - protein, 100 aa, Hadronyche versuta
MKFSKLSLTLALILTQALFVLCGKINEDFMENGLESHALHDEIRKPIDTEKADAERLVDCVVNTLGCSSDKDCCGMTPSCTLGICAPSVGLVGGLLGRAL

SEQ ID NO: 27 - protein, 97 aa, Atrax robustus
MKFSKLSITLAVILTQAVFVFCGMTNEDFMEKGFKSNDLQYAIKQPVNSGKPDTERLLDCVLSRVCSSDENCCGLTPTCTMGLCVPNVGGLLGGLLS

SEQ ID NO: 28 - protein, 97 aa, Atrax robustus
MKFSKLSITLVVILTQAVFVFCGMTNEDFMEKGFKSNDLQYAIRQPVNSGKPDTERLLDCVLSRVCSSDENCCGLTPTCTMGLCVPNVGGLLGGLLS

SEQ ID NO: 29 - protein, 101 aa, Hadronyche versuta
MKFSKLSLTLALILTQALLVLCGKINEDFMENGLESHALHDEIRKPLDTENPDTERQLDCVLNTLGCSSDKDCCGMTPSCTLGICAPNVGGLVGGLLGRAL

SEQ ID NO: 30 - protein, 101 aa, Hadronyche versuta
MKFSKLSLTLALILTQVLLVVCGKINEDFMENGLESHALHDEIRKPIDTEKADAERVLDCVVNTLGCSSDKDCCGMTPSCTLGICAPSVGGIVGGLLGRAL

SEQ ID NO: 31 - protein, 101 aa, Hadronyche versuta
MKFSKLSLTLALILAQAIFVLCGKINEDFMENGLESHALHDEIRKPIDTEKADAERVVDCVLNTLGCSSDKDCCGMTPSCTLGICAPSVGGLVGGLLGRAL

SEQ ID NO: 32 - protein, 101 aa, Hadronyche versuta
MKFSKLSLTLALILTQALLVVCGKINEDFMENGLESHALHDEIRKPIDTEKADAERVLDCVVNILGCSSDKDCCGMTPSCTLGICAPSVGGIVGGLLGRAL

SEQ ID NO: 33 - protein, 101 aa, Hadronyche versuta
MKFSKLSLTLALILTQALLVVCGKINEDFMENGLESHALHDEIRKPIDTEKADAERVLDCVVNTLGCSSDKDCCGMTPSCTLGICAPSVGGIVGGLLGRAL

SEQ ID NO: 34 - protein, 101 aa, Hadronyche infensa
MKFSKLSLTLALILTQALLVVCGKINEDFMENGLESHALHDEIRKSIDTEKADAERVLDCVVNTLGCSSDKDCCGMTPSCTLGICAPSVGGIVGGLLGRAL

SEQ ID NO: 35 - protein, 95 aa, Hadronyche versuta
MKFSKLSLTFALILTQTLLVLCDFMENGLESHALHDEIRKPIDTEKADAERVLDCVVNTLGCSSDKDCCGMTPSCTLGICAPSVGGLVGGLLGRA

SEQ ID NO: 36 - protein, 76 aa, Hadronyche versuta
MNTATGFIVLLVLATVLGGVEAGESHMRKDAMGRVRRQYCVPVDQPCSLNTQPCCDDATCTQERNENGHTVYYCRA

SEQ ID NO: 37 - protein, 39 aa, Hadronyche versuta
QYCVPVDQPCSLNTQPCCDDATCTQERNENGHTVYYCRA

SEQ ID NO: 38 - protein, 39 aa, Hadronyche versuta
GSCVPVDQPCSLNTQPCCDDATCTQERNENGHTVYYCRA

SEQ ID NO: 39 - protein, 75 aa, Hadronyche versuta
MNTATGFIVLLVLATILGGIEAGESHMRKDAMGRVRRQYCVPVDQPCSLNTQPCCDDATCTQERNENGHTVYYCR

SEQ ID NO: 40 - protein, 38 aa, Hadronyche versuta
QYCVPVDQPCSLNTQPCCDDATCTQERNENGHTVYYCR

SEQ ID NO: 41 - protein, 75 aa, Hadronyche versuta
MNTATGFIVLLVLATVLGGIEAGESHMRKDAMGRVRRQYCVPVDQPCSLNTQPCCDDATCTQELNENDNTVYYCR

SEQ ID NO: 42 - protein, 38 aa, Hadronyche versuta
QYCVPVDQPCSLNTQPCCDDATCTQELNENDNTVYYCR

SEQ ID NO: 43 - protein, 76 aa, Hadronyche versuta
MNTATGFIVLLVLATILGGIEAGESHMRKDAMGRVRRQYCVPVDQPCSLNTQPCCDDATCTQERNENGHTVYYCRA

SEQ ID NO: 44 - protein, 39 aa, Hadronyche versuta; identical to SEQ ID NO: 37

SEQ ID NO: 45 - protein, 76 aa, Hadronyche versuta
MNTATGFIVLLVLATVLGGIEAGESHMRKDAMGRVRRQYCVPVDQPCSLNTQPCCDDATCTQELNENANPVYYCRA

SEQ ID NO: 46 - protein, 39 aa, Hadronyche versuta
QYCVPVDQPCSLNTQPCCDDATCTQELNENANPVYYCRA

SEQ ID NO: 47 - protein, 76 aa, Hadronyche versuta
MNTTTGFIVLLVLATILGGIEAGESHMRKDAMGRVRRQYCVPVDQPCSLNTQPCCDDATCTQELNENDNTVYYCRA

SEQ ID NO: 48 - protein, 39 aa, Hadronyche versuta
QYCVPVDQPCSLNTQPCCDDATCTQELNENDNTVYYCRA

SEQ ID NO: 49 - protein, 76 aa, Hadronyche versuta
MNTATGFIVLLVLATVLGGIEAGESHMRKDAMGRVRRQYCVPVDQPCSLNTQPCCDDATCTQELNENDNTVYYCRA

SEQ ID NO: 50 - protein, 39 aa, Hadronyche versuta; identical to SEQ ID NO: 48

SEQ ID NO: 51 - protein, 76 aa, Hadronyche versuta
MNTATGFIVFLVLATVLGGIEAGESHMRKDAMGRVRRQYCVPVDQPCSLNTQPCCDDATCTQELNENDNTVYYCRA

SEQ ID NO: 52 - protein, 39 aa, Hadronyche versuta; identical to SEQ ID NO: 48

SEQ ID NO: 53 - protein, 76 aa, Hadronyche versuta
MNTATGFIVLLVLATVLGGIEARESHMRKDAMGRVRRQYCVPVDQPCSLNTQPCCDDATCTQELNENDNTVYYCRA

SEQ ID NO: 54 - protein, 39 aa, Hadronyche versuta; identical to SEQ ID NO: 48

SEQ ID NO: 55 - protein, 37 aa, Hadronyche versuta
SPTCIPSGQPCPYNENCCSQSCTFKENENGNTVKRCD

SEQ ID NO: 56 - protein, 45 aa, Hadronyche versuta
LLACLFGNGRCSSNRDCCELTPVCKRGSCVSSGPGLVGGILGGIL

SEQ ID NO: 57 - protein, 19 aa, Hadronyche versuta
EDTRADLQGGEAAEKVFRR

SEQ ID NO: 58 - protein, 15 aa, Hadronyche versuta
GESHVREDAMGRARR

SEQ ID NO: 59 - protein, 78 aa, Atrax robustus
MNTATGVIALLVLATVIGCIEAEDTRADLQGGEAAEKVFRRSPTCIPSGQPCPYNENCCSQSCTFKENENGNTVKRCD

SEQ ID NO: 60 - protein, 37 aa, Atrax robustus; identical to SEQ ID NO: 55

SEQ ID NO: 61 - protein, 78 aa, Atrax robustus
MNTATGVIALLVLVTVIGCIEAEDTRADLQGGEAAEKVFRRSPTCIPSGQPCPYNENCCSQSCTFKENENGNTVKRCD

SEQ ID NO: 62 - protein, 37 aa, Atrax robustus; identical to SEQ ID NO: 55

SEQ ID NO: 63 - protein, 78 aa, Atrax robustus
MNTATGVIALLVLATVIGCIEAEDTRADLQGGEAAEKVFRRSPTCIPSGQPCPYNENCCSQSCTFKENETGNTVKRCD

SEQ ID NO: 64 - protein, 37 aa, Atrax robustus
SPTCIPSGQPCPYNENCCSQSCTFKENETGNTVKRCD

SEQ ID NO: 65 - protein, 78 aa, Atrax robustus
MNTATGVIALLVLATVIGCIEAEDTRADLQGGEAAEKVFRRSPTCIPSGQPCPYNENCCSQSCTFKENENANTVKRCD

SEQ ID NO: 66 - protein, 37 aa, Atrax robustus
SPTCIPSGQPCPYNENCCSQSCTFKENENANTVKRCD

SEQ ID NO: 67 - protein, 78 aa, Atrax robustus
MNTATGVIALLVLATVIGCIEAEDTRADLQGGEAAEKVFRRSPTCIPSGQPCPYNENCCSKSCTYKENENGNTVQRCD

SEQ ID NO: 68 - protein, 37 aa, Atrax robustus
SPTCIPSGQPCPYNENCCSKSCTYKENENGNTVQRCD

SEQ ID NO: 69 - protein, 73 aa, Atrax robustus
MNTATGFIVLLVLATVLGCIEAGESHVREDAMGRARRGACTPTGQPCPYNESCCSGSCQEQLNENGHTVKRCV

SEQ ID NO: 70 - protein, 36 aa, Atrax robustus
GACTPTGQPCPYNESCCSGSCQEQLNENGHTVKRCV

SEQ ID NO: 71 - protein, 79 aa, Hadronyche versuta
MNTATGFIVLLVLATVIGCISADFQGGFEPYEGEDAERIFRRSPTCIPTGQPCPYNENCCSQSCTYKANENGNQVKGCD

SEQ ID NO: 72 - protein, 37 aa, Hadronyche versuta
SPTCIPTGQPCPYNENCCSQSCTYKANENGNQVKGCD

SEQ ID NO: 73 - protein, 79 aa, Hadronyche versuta
MNTATGFIVLLVLATVIGCISADFQGGFEPYEEEDAERIFRRSPTCIPTGQPCPYNENCCNQSCTYKANENGNQVKRCD

SEQ ID NO: 74 - protein, 37 aa, Hadronyche versuta
SPTCIPTGQPCPYNENCCNQSCTYKANENGNQVKRCD

SEQ ID NO: 75 - protein, 79 aa, Hadronyche versuta
MNTATGFIVLLVLATVIGCISADFQGGFEPYEEEDAERIFRRSPTCIPTGQPCPYNENCCSQSCTYKANENGNQVKRCD

SEQ ID NO: 76 - protein, 37 aa, Hadronyche versuta
SPTCIPTGQPCPYNENCCSQSCTYKANENGNQVKRCD

SEQ ID NO: 77 - protein, 79 aa, Hadronyche versuta
MNTATGFIVLLVLATVIGCISVDFQGGFESYEEEDAERIFRRSPTCIPTGQPCPYNENCCSQSCTYKANENGNQVKRCD

SEQ ID NO: 78 - protein, 37 aa, Hadronyche versuta; identical to SEQ ID NO: 76

SEQ ID NO: 79 - protein, 78 aa, Hadronyche versuta
MNTATGFIVLLVLATVIGCISADFQGGFESSVEDAERLFRRSSTCIRTDQPCPYNESCCSGSCTYKANENGNQVKRCD

SEQ ID NO: 80 - protein, 37 aa, Hadronyche versuta
SSTCIRTDQPCPYNESCCSGSCTYKANENGNQVKRCD

SEQ ID NO: 81 - protein, 78 aa, Hadronyche versuta
MNTATGFIVLLVLATVIGCISADFQGGFEPYEEEDAERIFRRSTCTPTDQPCPYHESCCSGSCTYKANENGNQVKRCD

SEQ ID NO: 82 - protein, 36 aa, Hadronyche versuta
STCTPTDQPCPYHESCCSGSCTYKANENGNQVKRCD

SEQ ID NO: 83 - protein, 78 aa, Hadronyche versuta
MNTATGFIVLLVLATVIGCISADFEGSFEPYEEEDAERIFRRSTCTPTDQPCPYDESCCSGSCTYKANENGNQVKRCD

SEQ ID NO: 84 - protein, 36 aa, Hadronyche versuta
STCTPTDQPCPYDESCCSGSCTYKANENGNQVKRCD

SEQ ID NO: 85 - protein, 78 aa, Hadronyche versuta
MNTATGFIVLLVLATVIGCISADFQGSFEPYEEEDAERIFRRSTCTPTDQPCPYDESCCSGSCTYKANENGNQVKRCD

SEQ ID NO: 86 - protein, 36 aa, Hadronyche versuta; identical to SEQ ID NO: 84

SEQ ID NO: 87 - protein, 78 aa, Hadronyche versuta
MNTATGFIVLLVLATVIGCISADFQGSFEPYEEEDAERIFRRSTCTPTDQPCPYHESCCSGSCTYKANENGNQVKRCD

SEQ ID NO: 88 - protein, 36 aa, Hadronyche versuta; identical to SEQ ID NO: 82

SEQ ID NO: 89 - protein, 78 aa, Hadronyche versuta
MNTATGFIVLLVLATVIGCISADFQGGFEPYEEEDAERIFRRSTCTPTDQPCPYDESCCSGSCTYKANENGNQVKRCD

SEQ ID NO: 90 - protein, 36 aa, Hadronyche versuta; identical to SEQ ID NO: 84

SEQ ID NO: 91 - protein, 36 aa, Atrax infensus; identical to SEQ ID NO: 82

SEQ ID NO: 92 - protein, 37 aa, Atrax infensus; identical to SEQ ID NO: 76

SEQ ID NO: 93 - protein, 37 aa, Atrax infensus; identical to SEQ ID NO: 80

SEQ ID NO: 94 - protein, 37 aa, Atrax robustus
SSVCIPSGQPCPYNEHCCSGSCTYKENENGNTVQRCD

SEQ ID NO: 95 - protein, 37 aa, Hadronyche versuta; identical to SEQ ID NO: 55

SEQ ID NO: 96 - protein, 37 aa, Atrax formidabillis
SPTCTGADRPCAACCPCCPGTSCKGPEPNGVSYCRND

SEQ ID NO: 97 - protein, 36 aa, Atrax formidabillis
SPTCTGADRPCAACCPCCPGTSCKGPEPNGVSYCRN

SEQ ID NO: 98 - protein, 37 aa, Atrax formidabillis
SPTCIRSGQPCPYNENCCSQSCTFKTNENGNTVKRCD

SEQ ID NO: 99 - protein, 9 aa, Atrax infensus
NGNQVKRCD

SEQ ID NO: 100 - protein, 9 aa, Atrax infensus
NGNQVKRCD

SEQ ID NO: 101 - protein, 9 aa, Hadronyche versutus
NGNTVKRCD

SEQ ID NO: 102 - protein, 15 aa, Hadronyche versutus
SPTCIPSGQPCPYNE

SEQ ID NO: 103 - protein, 16 aa, Agelenopsis aperta
ECVPENGHCRDWYDEC

SEQ ID NO: 104 - protein, 37 aa, Agelenopsis aperta
ECATKNKRCADWAGPWCCDGLYCSCRSYPGCMCRPSS

SEQ ID NO: 105 - protein, 38 aa, Agelenopsis aperta
ADCVGDGQRCADWAGPYCCSGYYCSCRSMPYCRCRSDS

SEQ ID NO: 106 - protein, 37 aa, Agelenopsis aperta
ACVGENQQCADWAGPHCCDGYYCTCRYFPKCICRNNN

SEQ ID NO: 107 - protein, 37 aa, Agelenopsis aperta
ACVGENKQCADWAGPHCCDGYYCTCRYFPKCICRNNN

SEQ ID NO: 108 - protein, 37 aa, Agelenopsis aperta
DCVGESQQCADWAGPHCCDGYYCTCRYFPKCICVNNN

SEQ ID NO: 109 - protein, 36 aa, Hololena curta
SCVGEYGRCRSAYEDCCDGYYCNCSQPPYCLCRNNN

SEQ ID NO: 110 - protein, 38 aa, Hololena curta
ADCVGDGQKCADWFGPYCCSGYYCSCRSMPYCRCRSDS

SEQ ID NO: 111 - protein, 70 aa, Androctonus australis Hector
KKNGYAVDSSGKAPECLLSNYCNNQCTKVHYADKGYCCLLSCYCFGLNDDKKVLEISDTRKSYCDTTIIN

SEQ ID NO: 112 - protein, 70 aa, Androctonus australis Hector
KKNGYAVDSSGKAPECLLSNYCNNECTKVHYADKGYCCLLSCYCFGLNDDKKVLEISDTRKSYCDTTIIN

SEQ ID NO: 113 - protein, 70 aa, Androctonus australis Hector
KKDGYAVDSSGKAPECLLSNYCYNECTKVHYADKGYCCLLSCYCFGLNDDKKVLEISDTRKSYCDTPIIN

SEQ ID NO: 114 - protein, 33 aa, Scorpio maurus palmatus
ALPLSGEYEPCVRPRKCKPGLVCNKQQICVDPK

SEQ ID NO: 115 - protein, 61 aa, Leiurus quinquestriatus
DGYIRKRDGCKLSCLFGNEGCNKECKSYGGSYGYCWTWGLACWCEGLPDEKTWKSETNTCG

SEQ ID NO: 116 - protein, 61 aa, Buthotus judaicus
DGYIRKKDGCKVSCIIGNEGCRKECVAHGGSFGYCWTWGLACWCENLPDAVTWKSSTNTCG

SEQ ID NO: 117 - protein, 39 aa, Atrax robustus
GSSPTCIPSGQPCPYNENCCSQSCTFKENENGNTVKRCD

SEQ ID NO: 118 - protein, 39 aa, Hadronyche Versuta
GSAICTGADRPCAACCPCCPGTSCKAESNGVSYCRKDEP

SEQ ID NO: 119 - protein, 41 aa, Hadronyche Versuta
GSQYCVPVDQPCSLNTQPCCDDATCTQERNENGHTVYYCRA

SEQ ID NO: 120 - protein, 60 aa, Hadronyche versuta
MNTATGFIVLLVLATVLGGVEAGESHMRKDAMGRVRRQYCVPVDQPCSLNTQPCCDDATC

SEQ ID NO: 121 - protein, 39 aa, Hadronyche versuta
QYCVPVDQPCSLNTQPCCDDAYCTQERNENGHTVYYCRA

SEQ ID NO: 122 - protein, 39 aa, Hadronyche versuta; identical to SEQ ID NO: 38

SEQ ID NO: 123 - protein, 74 aa, Hadronyche versuta
MNTATGFIVLLVLATILGGIEAGESHMRKDAMGRVRRQYCVPVDQPCSLNTQPCCDDATCTERNENGHTVYYCR

SEQ ID NO: 124 - protein, 38 aa, Hadronyche versuta; identical to SEQ ID NO: 40

SEQ ID NO: 125 - protein, 75 aa, Hadronyche versuta
MNTATGFIVLLVLATVLGGIEAGESHMRKDAMGRVRRQYCVPVDQPCSLNTQPCCDDAYCTQELNENDNTVYYCR

SEQ ID NO: 126 - protein, 38 aa, Hadronyche versuta; identical to SEQ ID NO: 42

SEQ ID NO: 127 - protein, 76 aa, Hadronyche versuta; identical to SEQ ID NO: 43

SEQ ID NO: 128 - protein, 39 aa, Hadronyche versuta; identical to SEQ ID NO: 37

SEQ ID NO: 129 - protein, 76 aa, Hadronyche versuta; identical to SEQ ID NO: 45

SEQ ID NO: 130 - protein, 39 aa, Hadronyche versuta; identical to SEQ ID NO: 46

SEQ ID NO: 131 - protein, 76 aa, Hadronyche versuta
MNTTTGFIVLLVLATILGGIEAGESHMRKDAMGRVRRQYCVPVDQPCSLNTQPCCDDAYCTQELNENDNTVYYCRA

SEQ ID NO: 132 - protein, 39 aa, Hadronyche versuta; identical to SEQ ID NO: 48

SEQ ID NO: 133 - protein, 76 aa, Hadronyche versuta; identical to SEQ ID NO: 49

SEQ ID NO: 134 - protein, 39 aa, Hadronyche versuta; identical to SEQ ID NO: 48

SEQ ID NO: 135 - protein, 76 aa, Hadronyche versuta; identical to SEQ ID NO: 51

SEQ ID NO: 136 - protein, 39 aa, Hadronyche versuta; identical to SEQ ID NO: 48

SEQ ID NO: 137 - protein, 76 aa, Atrax robustus; identical to SEQ ID NO: 53

SEQ ID NO: 138 - protein, 39 aa, Atrax robustus; identical to SEQ ID NO: 48

SEQ ID NO: 139 - protein, 22 aa, Hadronyche versuta
  MISC_FEATURE (4)..(4): Xaa can be any naturally occuring amino acid
MNTXTGFIVXLVLATXLGGXEA

SEQ ID NO: 140 - protein, 15 aa, Hadronyche versuta
  MISC_FEATURE (1)..(1): Xaa can be any naturally occuring amino acid
XESHMRKDAMGRVRR

SEQ ID NO: 141 - protein, 64 aa, Leiurus quinquestriatus hebraeus
VRDAYIAKNYNCVYECFRDAYCNELCTKNGASSGYCQWAGKYGNACWCYALPDNVPIRVPGKCR

SEQ ID NO: 142 - protein, 64 aa, Leiurus quinquestriatus quinquestriatus
VRDAYIAKNYNCVYECFRDSYCNDLCTKNGASSGYCQWAGKYGNACWCYALPDNVPIRVPGKCH

SEQ ID NO: 143 - protein, 65 aa, Bothus occitanus tunetanus
VRDAYIAQNYNCVYFCMKDDYCNDLCTKNGASSGYCQWAGKYGNACWCYALPDNVPIRIPGKCHS

SEQ ID NO: 144 - protein, 64 aa, Hottentotta judaica
GRDAYALDNLNCAYTCGSKSYCNTECTKNGAVSGYCQWLGKYGNACWCINLPDKVPIRIPGACR

SEQ ID NO: 145 - protein, 67 aa, Leiurus quinquestriatus hebraeus
VRDGYIAQPENCVYHCFPGSSGCDTLCKEKGGTSGHCGFKVGHGLACWCNALPDNVGIIVEGEKCHS

SEQ ID NO: 146 - protein, 66 aa, Buthus occitanus mardochei
GRDGYIAQPENCVYHCFPGSSGCDTLCKEKGATSGHCGFLPGSGVACWCDNLPNKVPIVVGGEKCH

SEQ ID NO: 147 - protein, 65 aa, Buthus occitanus mardochei
GRDAYIAQPENCVYECAKNSYCNDLCTKNGAKSGYCQWLGKYGNACWCEDLPDNVPIRIPGKCHF

SEQ ID NO: 148 - protein, 64 aa, Bothus martensii Karsch
VRDAYIAKPHNCVYECARNEYCNDLCTKNGAKSGYCQWVGKYGNGCWCIELPDNVPIRVPGKCH

SEQ ID NO: 149 - protein, 64 aa, Bothus martensii Karsch
VRDAYIAKPHNCVYSCARNEWCNDLCTKNGAKSGYCQWVGKYGNGCWCIELPDNVPIRVPGKCH

SEQ ID NO: 150 - protein, 64 aa, Bothus martensii Karsch
VRDAYIAKPENCVYHCAGNEGCNKLCTDNGAESGYCQWGGRYGNACWCIKLPDDVPIRVPGKCH

SEQ ID NO: 151 - protein, 66 aa, Bothus martensii Karsch
VRDGYIALPHNCAYGCLNNEYCNNLCTKDGAKIGYCNIVGKYGNACWCIQLPDNVPIRVPGRCHPA

SEQ ID NO: 152 - protein, 64 aa, Leiurus quinquestriatus
VRDGYIAQPENCVYHCIPDCDTLCKDNGGTGGHCGFKLGHGIACWCNALPDNVGIIVDGVKCHK

SEQ ID NO: 153 - protein, 66 aa, Leiurus quinquestriatus
VRDGYIAKPENCAHHCFPGSSGCDTLCKENGGTGGHCGFKVGHGTACWCNALPDKVGIIVDGVKCH

SEQ ID NO: 154 - protein, 66 aa, Centruroides noxius
KEGYLVDIKNTGCKYECLKLGDNDYCLRECKQQYGKGAGGYCYAFACWCTHLYEQAIVWPLPNKRC

SEQ ID NO: 155 - protein, 66 aa, Centruroides noxius
KEGYLVELGTGCKYECFKLGDNDYCLRECKARYGKGAGGYCYAFGCWCTQLYEQAVVWPLKNKTCR

SEQ ID NO: 156 - protein, 66 aa, Centruroides suffusus suffusus
KEGYLVSKSTGCKYECLKLGDNDYCLRECKQQYGKSSGGYCYAFACWCTHLYEQAVVWPLPNKTCN

SEQ ID NO: 157 - protein, 66 aa, Centruroides suffusus suffusus
KEGYLVNSYTGCKFECFKLGDNDYCLRECRQQYGKGSGGYCYAFGCWCTHLYEQAVVWPLPNKTCN

SEQ ID NO: 158 - protein, 76 aa, Buthotus judaicus
KKNGYPLDRNGKTTECSGVNAIAPHYCNSECTKVYVAESGYCCWGACYCFGLEDDKPIGPMKDITKKYCDVQIIPS

SEQ ID NO: 159 - protein, 70 aa, Androctonus australis Hector; identical to SEQ ID NO: 112

SEQ ID NO: 160 - protein, 70 aa, Leiurus quinquestriatus quinquestriatus
KKNGYAVDSSGKAPECLLSNYCYNECTKVHYADKGYCCLLSCYCVGLSDDKKVLEISDARKKYCDFVTIN

SEQ ID NO: 161 - protein, 70 aa, Leiurus quinquestriatus hebraeus
KKNGFAVDSNGKAPECFFDHYCNSECTKVYYAEKGYCCLLSCYCFGLNDDKKVLEISDTTKKYCDFTIIN

SEQ ID NO: 162 - protein, 72 aa, Bothus martensii Karsch
KKNGYAVDSSGKVAECLFNNYCNNECTKVYYADKGYCCLLKCYCFGLLDDKPVLDIWDSTKNYCDVQIIDLS

SEQ ID NO: 163 - protein, 61 aa, Leiurus quinquestriatus hebraeus
DGYIKRRDGCKVACLIGNEGCDKECKAYGGSYGYCWTWGLACWCEGLPDDKTWKSETNTCG

SEQ ID NO: 164 - protein, 60 aa, Leiurus quinquestriatus hebraeus
DGYIRGDGCKVSCVINHVFCDNECKAAGGSYGYCWAWGLACWCEGLPAEREWKYETNTCG

SEQ ID NO: 165 - protein, 62 aa, Buthotus judaicus
DGYIRKKDGCKVSCIIGNEGCRKECVAHGGSFGYCWTWGLACWCENLPDAVTWKSSTNTCGR

SEQ ID NO: 166 - protein, 61 aa, Buthacus arenicola
DGYIRRRDGCKVSCLFGNEGCDKECKAYGGSYGYCWTWGLACWCEGLPDDKTWKSETNTCG

SEQ ID NO: 167 - protein, 61 aa, Leiurus quinquestriatus quinquestriatus
DGYIRKRDGCKLSCLFGNEGCNKECKSYGGSYGYCWTWGLACWCEGLPDDKTWKSETNTCG

SEQ ID NO: 168 - protein, 60 aa, Bothus occitanus tunetanus
DGYIKGYKGCKITCVINDDYCDTECKAEGGTYGYCWKWGLACWCEDLPDEKRWKSETNTC

SEQ ID NO: 169 - protein, 63 aa, Leiurus quinquestriatus hebraeus
DNGYLLNKATGCKVWCVINNASCNSECKLRRGNYGYCYFWKLACYCEGAPKSELWAYATNKCN

SEQ ID NO: 170 - protein, 62 aa, Tityus serrulatus
KEGYLMDHEGCKLSCFIRPSGYCGRECGIKKGSSGYCYAWPACYCYGLPNWVKVWDRATNKC

SEQ ID NO: 171 - protein, 64 aa, Tityus zulianus
KDGYLVGNDGCKYSCFTRPGTYCANECSRVKGKDGYCYAWMACYCYSMPNWVKTWDRATNRCGR

SEQ ID NO: 172 - protein, 29 aa, Oldenlandia affinis
GLPVCGETCVGGTCNTPGCTCSWPVCTRN

SEQ ID NO: 173 - protein, 29 aa, Oldenlandia affinis
CGETCFGGTCNTPGCSCTWPICTRDGLPV

SEQ ID NO: 174 - protein, 30 aa, Oldenlandia affinis
GTPCGESCVYIPCISGVIGCSCTDKVCYLN

(19) United States
(12) Patent Application Publication
(10) Pub. No.: US 2017/0121377 A1
(43) Pub. Date: May 4, 2017

(54) ARTIFICIALLY ACTIVATED TOXIC PEPTIDES

(71) Applicant: Vestaron Corporation, Kalamazoo, MI (US)

(72) Inventors: Robert M. Kennedy, Dexter, MI (US); Lin Bao, Portage, MI (US); Alvar R. Carlson, Kalamazoo, MI (US); Catherine L. Foune, Gobles, MI (US); Alexandra M. Haase, Martin, MI (US); Bruce A. SteinBaugh, Portage, MI (US)

(73) Assignee: Vestaron Corporation, Kalamazoo, MI (US)

(21) Appl. No.: 15/301,030

(22) PCT Filed: Apr. 3, 2015
(86) PCT No.: PCT/US2015/024334

§ 371 Date: Sep. 30, 2016

Related U.S. Application Data
(60) Provisional application No. 61/975,147, filed on Apr. 4, 2014.

(51) Int. Cl. C07K 14/435

(52) U.S. Cl. CPC C07K 14/43518

(57) ABSTRACT

Described are the artificially induced conversion of certain toxic peptides to create both different forms of those peptides and new and useful derivatives of the original peptides that are both useful by themselves and useful as new compounds and new stable intermediates that may be used to make other important compounds.

### CROSS REFERENCE TO RELATED APPLICATIONS

This application claims priority to U.S. Patent Application No. 61/975,147, filed Apr. 4, 2014, the entire contents of which are incorporated herein by reference.

### FIELD OF THE INVENTION

This invention relates to chemical and mechanical methods to increase the activity of natural and hybrid physiologically active peptides such as peptide toxins related to, or inspired from, the toxins found in venomous spiders, snails, mollusks and other animals.

### SEQUENCE LISTING

This application incorporates in its entirety the Sequence Listing entitled “FAM_N_PRV_SEQ_LISTING_2015_04_03_5T25.txt” (106,014 bytes), which was created on Apr. 3, 2015, and filed electronically herewith.

### BACKGROUND

Typically high heat and pressure, such as the conditions produced by autoclaves and used for sterilization, are used to neutralize and inactivate biological samples like fungi, bacteria and viruses. Often proteins are denatured or even destroyed by such a process. Usually when organisms are exposed to high temperatures and pressures, they fail to thrive or even survive because their proteins are denatured and consequently the organisms become inactive and die. The only biological process that follows is decay. Acidic conditions alone can sometimes produce a similar result. Expose most active peptides, like toxic proteins, to low pH or acid conditions and the peptide denatures and no longer functions like the native peptide or protein. Autoclaves are often used by medical offices to treat instruments, devices to make them safe and sterile for reuse and increasingly they are used to treat biologically contaminated waste to turn it into safe neutral harmless waste for disposal. Here we report the artificially induced conversion of certain toxic peptides to create both different forms of those peptides and new and useful derivatives of the original peptides that are both useful by themselves and useful as new compounds and new stable intermediates that useful to make other important compounds.

### SUMMARY OF THE INVENTION

This invention has two parts. In Part 1 we describe a process of using artificially induced chemical and mechanical methods to increase the activity and toxicity of a peptide, including a toxic peptide comprising the following steps, optionally in the letter order: a) mix said peptide with water to make an aqueous solution or aqueous emulsion of said peptide in a liquid or semi-liquid form, wherein the aqueous solution or aqueous emulsion is comprised of at least 10% water; b) measure the pH of said peptide in the aqueous solution or aqueous emulsion; c) adjust the pH of said solution or emulsion to less than pH 7.0. The pH may be between about 1.0 and about 6.5, between about 2.0 and about 6.0, between about 2.5 and about 5.5, between about 3.0 and about 5.0, between about 3.0 and about 4.0, about 3.2, 3.4, 3.5, 3.6, or 3.8.

The process wherein after said pH adjustment the peptide is dried to a dry powder or granular form. The pH adjustment can be made using a strong or weak acid. Strong acid examples are any of the following acids—chloric acid (HClO₃), hydrochloric acid (HCl), hydrobromic acid (HBr), hydroiodic acid (HI), phosphoric acid (H₃PO₄), sulfuric acid (H₂SO₄). Perchloric acid (HClO₄), and Nitric acid (HNO₃). Weak acid examples are acetic acid and/or oxalic acid. During the pH adjustment, the aqueous solution or aqueous emulsion is exposed to a dry heat i.e. a temperature increase without steam or pressure or heat, pressure and steam. Heat and heat and pressure conditions described in the specification can also be used with any of the procedures including the dry powder procedures described herein.

The process of removing any one or more covalently bound 2H+O or molecules from a peptide while said peptide is in an aqueous solution or emulsion by the reduction of the pH of the solution or emulsion to less than 7.0. The peptides that work especially well with the process are the peptides described in the specification or in the sequence listing and particularly SEQ ID NO. 119 and SEQ ID NO. 121.

In addition to the process we describe insecticidal compositions of the peptides and formulations suitable for application to the locus of an insect to be treated with the peptide. In addition to the process and compositions we describe toxic peptides per se, with any one or more covalently bound 2H+O or molecules removed pH of the peptide in aqueous solution or emulsion is reduced to less than 7.0.

We describe a process of increasing the toxicity and/or activity of a peptide, comprising the following steps: a)prepare said peptide as a pure Form 1 peptide, or peptide acid or composition containing less than about 10% water, b) place said Form 1 peptide in a controllable chamber or heating platform; c) heat said peptide to a desired temperature, with or without pressure, with or without steam; d) maintain the heated peptide at the desired temperature, pressure and steam until the desired amount of Form 1 peptide, called peptide acid, Converts to Form 2 peptide, called peptide lactone. The controllable chamber can maintain temperatures from 0 to 500° C. and pressures from atmospheric to 500 psi. The peptide can be heated to about the following temperatures; heated to at least about 10° C. but to no more than a maximum temperature selected from about 200° C., 300° C., or at most 400° C.

We describe a process where the peptide is: heated to at least from a temperature selected from about any of the following temperatures, temperature ranges or combinations of ranges of temperatures: 10° C. to 20° C.; 20° C. to 30° C.; 30° C. to 40° C.; 40° C. to 50° C.; 50° C. to 60° C.; 60° C. to 70° C.; 70° C. to 80° C.; 80° C. to 90° C.; 90° C. to 100° C.; 100° C. to 110° C., 110° C. to 120° C., 120° C. to 130° C., 130° C. to 140° C., 140° C. to 150° C., 150° C. to 160° C., 160° C. to 170° C., 170° C. to 180° C., 180° C. to 190° C., 190° C. - 200° C., 200° C. to 210° C., 210° C. to 220° C., 220° C. to 230° C., 230° C. to 240° C., 240° C. to 250° C., 250° C. to 260° C., 260° C. to 270° C., 270° C. to 280° C., 280° C. to 290° C., 290° C. to 300° C., 300° C. to 400° C. and 400° C. to 500° C.

We describe a process where the peptide, or peptide acid is exposed to any of the following pressures or ranges of pressures: a) from about 10 psi to about 40 psi; b) from about 15 psi to about 35 psi; c) from about 18 psi to about 25 psi; d) about 21 psi. The chosen temperature and pressure range from the following periods depending on the temperature and pressure chosen: a) from about 5 minutes to about 40 minutes; b) from about 10 minutes to about 30 minutes; c) from about 15 minutes to about 25 minutes; d) about 21 minutes.

The following conditions may be used, the peptide should be maintained at the following temperatures and pressures and times: a) between from about 100° C. to about 140° C.; at a pressure of from about 10 psi to about 40 psi; for from about 5 minutes to about 40 minutes; b) between from about 110° C. to about 130° C.; at a pressure of from about 15 psi to about 35 psi; for from about 10 minutes to about 30 minutes; c) between from about 115° C. to about 125° C.; at a pressure of from about 18 psi to about 25 psi; for from about 15 minutes to about 25 minutes; d) of about 121° C., at a pressure of about 21 psi, for about 20 minutes. In cases the pressure is no greater than atmospheric pressure and the temperature is selected from the temperatures of at least 50° C. to 60° C. or greater. In some cases the following temperatures, temperature ranges or combinations of ranges of temperatures are used: 50° C. to 60° C.; 60° C. to 70° C.; 70° C. to 80° C.; 80° C. to 90° C.; 90° C. to 100° C.; 100° C. to 110° C., 110° C. to 120° C., 120° C. to 130° C., 130° C. to 140° C., 140° C. to 150° C., 150° C. to 160° C., 160° C. to 170° C., 170° C. to 180° C., 180° C. to 190° C., 190° C. −200° C., 200° C. to 210° C., 210° C. to 220° C., 220° C. to 230° C., 230° C. to 240° C., 240° C. to 250° C., 250° C. to 260° C., 260° C. to 270° C., 270° C. to 280° C., 280° C. to 290° C., 290° C. to 300° C., 300° C. to 400° C. and 400° C. to 500° C.

The process may use the following temperatures and times, where the peptide is a) heated and maintained at a temperature of more than about 100° C. for at least about 1 hr.; b) heated and maintained at a temperature of between about from 80° C. to about 120° C. for at least about 2 hr.; c) heated and maintained at a temperature of between about from 50° C. to about 80° C. for at least about 3 hr. Alternatively the peptide may be a) heated and maintained at a temperature of more than about 180° C., and a pressure of at least about 5 psi for at least about 5 minutes; b) heated and maintained at a temperature of more than about 100° C., and a pressure of at least about 10 psi for at least about 10 minutes; c) heated and maintained at a temperature of between about from 80° C. to about 120° C., and a pressure of at least about 10 psi, for at least about 30 minutes.; or d) heated and maintained at a temperature of between about from 50° C. to about 80° C. for at least about 1 hr.

The peptide may be converted using the following conditions: a) heated and maintained at a temperature of between about 200° C. to about 300° C., and a pressure of between about 5 to about 10 psi for between about 5 to about 10 minutes; b) heated and maintained at a temperature of between about 150° C., and about 200° C., and a pressure of between about 10 to about 30 psi for between about 5 to about 30 minutes; c) heated and maintained at a temperature of between about from 80° C. to and about 150° C., and a pressure of between about 10 to about 20 psi for between about 20 to about 60 minutes; or d) heated and maintained at a temperature of between about from 50° C. to about 80° C. and a pressure of between about 10 to about 40 psi for between about 30 to about 60 minutes.

Alternative conditions are where the peptide is a) heated and maintained at a temperature of between about 110° C., and about 130° C., and a pressure of between about 10 to about 20 psi for between about 10 to about 20 minutes; or b) heated and maintained at a temperature of about 121° C., and a pressure about 21 psi for about 20 minutes.

In general we describe a process of removing any one or more covalently bound 2H+O, or H₂O or molecules from a peptide by the heating of said peptide under any of the conditions, temperatures and pressures as described herein. A process of removing any one or more covalently bound 2H+O, or H₂O or molecules from any peptide in the sequence listing by the heating of said peptide under any of the conditions, temperatures and pressures as described herein. We describe any peptide in the sequence listing after Conversion. We describe the peptides produced from any of the procedures described in the specification or claims. We describe insecticidal composition of the peptides produced by any of the processes of claims in a formulation suitable for application to the locus of an insect to be treated with the peptide. We describe a toxic peptide, and call it a peptide lactone when any one or more covalently bound 2H+O or molecules removed when the peptide is heated to any of the conditions, temperatures and pressures as described herein. We describe a toxic peptide described in any or produced by any of the procedures here where one or more covalently bound 2H+O or H₂O molecules removed, and then it is called a peptide lactone, herein and in Part 2.

Especially suitable conditions for conversion are to heat the peptide and maintain it at a temperature of about 121° C., and a pressure about 21 psi for about 20 minutes.

In Part 2 of this application we describe how the peptide lactone can be converted into a peptide hydrazide and the peptide hydrazide converted into a peptide hydrazone. We describe the process of making, and the peptide hydrazide product made by the process of converting an insect predator peptide from the peptide lactone form to the peptide hydrazide form comprising mixing an insect predator peptide lactone with hydrazine and purifying to obtain the peptide hydrazide. We describe how a peptide lactone is prepared in water, hydrazine monohydrate is added and the mixture is stirred to form the peptide hydrazide which is optionally frozen, thawed and purified to obtain purified peptide hydrazide. If desired the insect predator peptide can vary in size from about 20 amino acids to about 50 amino acids and has 2, 3 or 4 cystine bonds, or alternatively it has 3 or 4 cystine bonds or 2 or 3 cystine bonds. The peptide lactone can be prepared from any peptide in the sequence listing and any peptide in the sequence listing or any peptide with more than 80% homology to any peptide in the sequence listing, or any sequence having more than 85%, 90%, 95% or 99% homology and 3 or 4 cystine bonds.

We have demonstrated how to use these methods with the peptide named the Hybrid +2 peptide wherein either method a or method b can be used, comprising: method a; a) start with a solution of 100 mg of purified Form 2 peptide, the Hybrid+2 peptide lactone, in 1 mL of water, b) treat the 1 mL of 100 mg peptide lactone with 100 uL of hydrazine monohydrate and stir at room temperature to form the peptide hydrazide, optionally for 2 hours, c) purify the solution of peptide hydrazide on a prep HPLC (eluted with a gradient of acetonitrile/water/trifluoroacetic acid), d) select appropriate fractions of peptide hydrazide, e) combine appropriate fractions of peptide hydrazide and concentrating under vacuum to reduce the volume, f) freeze the reduced volume of peptide hydrazide, at below zero temperature, optionally at −80° C., g) freeze-dry the Hybrid +2 peptide hydrazide, optionally on a lyopholizer, to obtain Hybrid +2 peptide hydrazide (I); or method b, wherein method b comprises: a) stir a solution of 25 mL of Super Liquid Concentrate, which is a mixture of Form 1, the peptide acid and Form 2, optionally at about 50° C. to 90° C., optionally at 75° C., b) let the solution cool, c) treat solution with hydrazine monohydrate, optionally 2mL, and stir, optionally at room temperature for 2 hours d) purify portions on a prep HPLC, optionally eluted with a gradient of (acetonitrile/water/trifluoroacetic acid) e) combine and concentrate fractions, reduce volume, optionally under vacuum, f) freeze remaining liquid, optionally freeze at −80° C. and lyopholize to produce Hybrid +2 peptide hydrazide.

We also show how to use the peptide hydrazide and react it with a carbonyl to make a useful peptide hydrazone. This is done by converting an insect predator peptide from the peptide hydrazide to the peptide hydrazone comprising, a) mix a solution of hydrazide in water and add hexanal in ethanol, stir, b) treat with a stock solution made of hexanal, acetic acid and ethanol, stir, c) add a stock solution made from hexanal, acetic acid and ethanol, d) mix, let stand and then optionally heat to produce the hydrazone. We used this process to make Hydrazone (II). This was done by a) mixing a solution hydrazide (I) in water with hexanal in ethanol, stir, b) add some stock solution of claim 16, d) mix and let stand then optionally heat to produce Hydrazone (II).

The hydrazone is both a key stable intermediate and can also be a final product. The product being a pegylated peptides or PEG peptide. The hydrazone can be other things as well but we believe that it is most useful when it is pegylated. We also show an alkylated hydrazone. The pegylated peptide actually takes the form of a hydrazone. See Example 9 and Hydrazone (III) and Example 11 and (IX). Compounds like this have never existed before and the chemistry to make them has never been taught before. These peptide hydrazones are novel, the pegylated peptide hydrazones like Hydrazone (IX) are novel in two aspects. First, the unsaturated carbonyl linkage shown in Examples 10(b) and 11(b) have never been used before to link a PEG with a peptide. Second, starting this reaction with the aldehyde or ketone on the “pegylation side” that is where the aldehyde or ketone is bound to PEG and then reacting that with the peptide hydrazide has never been shown before. Usually the aldehyde or ketone is put on the peptide and then the peptide ketone or peptide aldehyde is reacted or combined with the PEG. Using an unsaturated carbonyl in this reaction makes the bond more stable and harder to break because the imine nitrogen is less basic. So a comparison can be made of the carbonyl in Example 9 where PEG is joined to the peptide with a saturated carbonyl with Example 11 where PEG is joined to the peptide with an unsaturated carbonyl. The unsaturated carbonyl linkage of Example 11 is especially important because it forms a stronger bond making a more durable linkage between the peptide and PEG. This stronger bond is the result of the unsaturated carbonyl making the imine nitrogen less basic and not as readily protonated which is the first step in hydrolysis of the hydrazone linkage. These types of bonds have never been used before to link peptides and PEG or alkyl groups.

Pegylated peptides are well known but this method of making them, from a peglated hydrazone made from a peptide lactone that is converted to a hydrazide is novel and unknown until now. The pegylated toxic insecticidal peptides are extremely important because when these insecticides are delivered to the insect via ingestion of plants, oral bioavailability is critically important. In a way this is very similar to how important oral bioavailability is to for a drug taken by a human when taken by mouth. In both situations the factor that controls how well the medicine “works” is its oral bioavailability. Pegylation of proteins increases the size and molecular weight of molecules. Pegylation decreases cellular protein clearance by reducing elimination through the retiduloendothelial system or by specific cell-protein interactions. In addition, pegylation forms a protective ‘shell’ around the protein. This shell and its associated waters of hydration shield the protein from immunogenic recognition and increase resistance to degradation by proteolytic enzymes, such as trypsin, chymotrypsin and Streptomyces griseus protease. See, Pegylation A Novel Process of Modifying Pharmacokinetics. J. Milton Harris, Nancy E. Martin and Marlene Modi, in Clin Pharmacolomry 2001; 40(7): 539-551 at 543. Pegylation increases bioavailability by giving the peptide a greater half life. For example, pegylation reduced the degradation of asparaginase by trypsin: after a 50 minute incubation period, there was 5, 25 and 98% residual activity of native asparaginase, PEG-asparaginase and branched-PEG-asparaginase, respectively. Id.

We show how to convert an insect predator peptide from the peptide lactone to the peptide hydrazide and finally to the peptide hydrazone, which which is a pegylated peptide. We give an example of the Peptide Hydrazide (I) mixing with an Aldehyde (IV) to make Peptide Hydrazone (III), a pegylated protein. The process involves, acidifying complex glycols with a strong or weak acid, adding hydrazide and mixing well to make peptide hydrazone. The peptide hydrazone can be a pegylated peptide depending on the carbonyl used to make the hydrazone. We show how to make the peptide Hydrazone (III) by a) adding 1 drop of acetic acid to a stock solution of the mixture of compounds referred to as O-[2-(6-Oxocaproylamino)ethyl]-O′-methylpolyethylene glycol (IV) in ethanol, b) use the stock solution of O-[2-(6-Oxocaproylamino)ethyl]-O′-methylpolyethylene glycol (IV) (MW˜2′000) treated with acetic acid from step a and add it to a solution of hydrazide (I) in water, c) mix and allow to stand at room temperature, d) add the remainder of the stock solution of O-[2-(6-Oxocaproylamino)ethyl]-O′-methylpolyethylene glycol (IV)(MW˜2′000) in portions and allow the mixture to stand overnight after mixing to produce Peptide Hydrazone (III). We show how an insect predator peptide hydrazide can be converted to the peptide hydrazone comprising, adding an acrylic ketone to a hydrazide to make a hydrazone. The latter process is demonstrated with the process for making the peptide Hydrazone (VI) comprising, adding acrylic ketone (V) in ethanol to a solution of hydrazide (I) in water and mixing. We also make the peptide Hydrazone (IX) comprising adding PEG4 Ketone (VIII) to a solution of hydrazide (I) in water, and mixing to make Hydrazone (IX). The peptide hydrazone is thus shown to be a key intermediate needed to make the pegylated peptides according to our process.

We describe a process of preparing a peptide and or the peptide produced by the process and or an insecticidal composition produced by the process described as removing any one or more covalently bound 2H+O, or H₂O or molecules from a peptide; including any toxic peptide with any one or more covalently bound 2H+0 or molecules removed under any of the conditions, temperatures, pressures and pH or acidic conditions, either alone or in combination as described herein or found in the specification or claims including any of the peptides, hydrazides or hydrazones produced from any of the procedures described in the specification and claims or use of any of these peptides as insecticidal compositions of the peptides produced by any of the processes described in the specification and claims and then used in a formulation suitable for application to the locus of an insect.

### BRIEF DESCRIPTION OF THE FIGURES

FIG. 1 is a Mass Spec. of SEQ ID NO: 119, with an arrow showing Peak 1 has the number 11.84.

FIG. 2 is a Mass Spec. of SEQ ID NO: 119, with a deconvoluted spectrum of Peak 1, shown in FIG. 1, where the deconvoluted Peak 1 of FIG. 1, has the value 4562.8896.

FIG. 3 is a Mass Spec. of SEQ ID NO: 119 with an arrow showing Peak 2 has the number 12.82.

FIG. 4 is a Mass Spec. of SEQ ID NO: 119, with a deconvoluted spectrum of Peak 2 shown in FIG. 3, having the mass value 4544.8838.

FIG. 5 is a bar graph that shows a comparison of the toxicity of the peptide of the original form, Peak 1, compared to the toxicity of the peptide of the new form, after treatment, i.e. Peak 2. Both forms are also compared to a control.

FIG. 6 is a bioassay comparison of Peak 1 and Peak 2 separately prepared from liquid chromatography. Peak 1 results are shown.

FIG. 7 is a bioassay comparison of Peak 1 and Peak 2 separately prepared from liquid chromatography. Peak 2 results are shown.

FIG. 8 is a Mass Spec. of SEQ ID NO: 119 at pH 5.6 from the Stability pH Study

FIG. 9 is a Mass Spec. of SEQ ID NO: 119 at pH 3.9 from the Stability pH Study

FIG. 10 is a Mass Spec. of SEQ ID NO: 119 at pH 8.3 from the Stability pH Study

FIG. 11 shows Peaks 1, 2 and 3 from HPLC and it shows that H₂O and NH₃ can be separately lost from SEQ ID NO: 121, or native hybrid, upon heating. Three HPLC peaks, of which UV absorbance changed with temperature, have been identified at retention time of 4.2 min, 5.4 min and 6.9 min.

FIG. 12 shows the results of a TOF MS Evaluation of the isoforms of the native hybrid peptide.

FIG. 13 is a Mass Spec. of Hydrazide (I).

FIG. 14 is a Mass Spec. of Hydrazide (I), with a deconvoluted spectrum.

FIG. 15 is a Mass Spec. of Hydrazone (II).

FIG. 16 is a Mass Spec. of Hydrazone (II), with a deconvoluted spectrum.

FIG. 17 is a Mass Spec. of Hydrazone (III).

FIG. 18 is a Mass Spec. of Hydrazone (III), with the molecular ions seen showing a distribution.

FIG. 19 is a Mass Spec. of Acrylic Ketone (V), UV trace.

FIG. 20 is a Mass Spec. of Acrylic Ketone (V).

FIG. 21 is a Mass Spec. of Hydrazone (VI).

FIG. 22 is a Mass Spec. of Hydrazone (VI), with a deconvoluted spectrum.

FIG. 23 is a Mass Spec. of PEG4 Ketone (VIII), UV trace.

FIG. 24 is a Mass Spec. of PEG4 Ketone (VIII).

FIG. 25 is a Mass Spec. of Hydrazone (IX).

FIG. 26 is a Mass Spec. of Hydrazone (IX), with a deconvoluted spectrum.

### DETAILED DESCRIPTION OF THE INVENTION

### Definitions

The definitions should be read and understood in view of the application as a whole, its descriptions, examples and claims.

AI means active ingredient.

Autoclave means a device, with a pressure vessel that can be closed or locked and that allows for the addition of steam and or heated water, typically allowing for the removal of dry air with steam, sometimes with vacuum pumps, optionally allowing for steam pulsing or cycling in order to produce higher temperatures either with dry heat and/or with high pressure and optionally steam, if desired. It usually powered from an attached electric cord, a power cord, that carries current from a wall outlet to the device to power the heat and pressure made by the device, but it can refer to a simple pressure vessel that could be heated on a stove top.

Carbonyl means an aldehyde or ketone.

Chamber means an enclosed vessel or space.

Centigrade is a unit of temperature, usually as degree, it may be abbreviated C as in 40 C or ° C. as in 40° C.

Convert and Conversion means the transformation of a peptide from what is described as Form 1 to Form 2, using the methods described herein of heat, heat and steam and/or pressure or acid conditions either alone or in combination with other factors. Conversion is more fully described and exemplified herein.

DI means deionized water.

Form 1 or Form 1 peptide, refers to the form of a peptide, form suggesting the way it is folded or presents its active sites and its number or degree of internal bonding, and specifically Form I or Form 1 means a peptide as it exists when it is first formed and without the loss of 2H plus O or 18 daltons from its molecular weight. Form 1 is also known as the acid form of the peptide sometimes called here the peptide acid.

Form 2 or Form 2 peptide, refers to the form of a peptide, form suggesting the way it is folded or presents its active sites and its number or degree of internal bonding, and specifically Form II or Form 2 means a peptide that began as Form 1 peptide but was transformed through the application of any one of a combination of treatments described herein such as: heat, temperatures, pressure, steam, acid, low pH conditions resulting in the loss of a 18 daltons equivalent to a water molecule, when measured before and after it Converts from Form 1 to Form 2. When a peptide begins in one form and then looses 2H plus O or 18 daltons from its molecular weight it then exists as a Form 2 peptide. Form 2 is also known as the lactone form of the peptide or peptide lactone. See the first paragraph in Part 2 for the definition of lactone, as it is used in this document.

Formulation means a mixture of ingredients usually including the active ingredient, here typically a toxic peptide with other ingredients to increase the solubility, stability, spreadability, effectiveness, safety or other desired properties usually associated with storing or delivering the active ingredient.

Insect and Insect to be treated means an insect that a person having knowledge of the insect would like the insect controlled in some fashion such as limiting its food consumption, limiting its growth or shortening its life because it is perceived to consume or destroy food or materials or by it nature and presence it is undesirable.

Locus of an insect means the place where an insect normally lives, eats, sleeps or travels to or from.

Physiologically active peptide means a toxic peptide that is biologically active.

Pressure vessel means an enclosed container capable of holding a high pressure, with dry or wet pressured device that can, with the addition of water, produce heated steam and high temperatures. A pressure vessel needs to receive power from an external source, such as from a stove top heating ring, or as part of a autoclaved device.

Strong acid means an acid that ionizes completely in a solution of water. It has a low pH, usually between 1 and 3. Examples include: hydrochloric acid—HCl, hydrobromic acid—HBr, hydroiodic acid—HI, sulfuric acid—H₂SO₄, phosphoric acid (H₃PO₄), perchloric acid HClO₄, nitric acid HNO₃ and chloric acid HClO₃.

Toxic peptide means a peptide, natural, artificial or synthetic, composed of amino acids, natural or artificial that produces harmful effect on insects when they are exposed to the peptides. Toxic peptides includes venomous peptides which are peptides from or related to venomous creatures like spiders, snakes, molluscs and snails. Toxic peptides includes the peptides identified and described in U.S. Pat. No. 8,217,003 and U.S. Pat. No. 8,501,684.

Water about 10% or a least about 10% or 10% or more or less means any formulation or mixture than has at least about 10% of its total weight or amount, available as water, that is water molecules not covalently bound as part of a larger molecule and capable of ionization of the H₂O molecules, that is capable of maintaining a pH.

Weak acid means an acid that does not dissociate completely when in a water solution. They usually have a pH between 3 and 6. Examples include: acetic acid and oxalic acid. Weak acids exist in equilibrium between molecules that are ionized and those that are not.

### General Descriptions and Procedures

Described herein are various treatments including heat alone, heat in combination with heated water, steam, heat and pressure and/or independently acid treatments that can increase the activity of some peptides by nearly 5 times greater activity than before they were treated. Instead of losing their activity under high temperature and pressure, the activity of these peptides showed a dramatic increase in activity. We show the nature of the changes and the conditions and ranges of temperature, pressure and pH that can be used to increase, rather than decrease, the activity of some peptides including the peptides we call physiologically active and or toxic peptides.

These peptides undergo what is essentially a dehydration by rearrangement process. We call this transformation “Conversion.” Conversion happens when a normally toxic peptide is transformed into a much more active and more toxic peptide using elevated temperature, or heat, with or without steam and pressure, or acid, or heat with acid, or acid with heat plus steam and/or pressure or various combinations of temperature, heat, heat with pressure, heat with steam and pressure, acidity or low pH, acid or low pH with heat, acid or low pH with heat and pressure, acid or low pH with heat, steam and pressure. Conversion can be made to occur relatively quickly when heat is applied or if the peptides are in water, when low pH is applied to an aqueous solution of peptides. A temperature increase, that is heat, with or without an increase in pressure; with or without steam; or a decrease in pH, that is by applying an acid or acidic conditions to liquid formulation; or a combination of both temperature and acid results in a surprising increase in the activity of certain toxin peptides that are described herein. Further observations, measurements and analysis of various embodiments related to this discovery are disclosed and claimed.

In some embodiments, peptides, toxic to insects, are treated with the following conditions: heat alone or heat in combination with steam and pressure, such as in a typical autoclave, operating at about 100° C. to 150° C. If steam and pressure are used with a pressure of about 100 kPa or 15 psi. for anywhere from 3, 5, 10, 20, 30, 40, 50, 60, 70, 75, 80 or 90 minutes depending on the variables of temperature, pressure and acidity then Conversion will result in a relatively short period of time. Suitable conditions for conversion are to heat the peptide and maintain it at a temperature of about 121° C., and a pressure about 21 psi for about 20 minutes. Some of the procedures described herein, in some embodiments, are similar to standard procedures used when autoclaving biological samples for reuse or safe disposal.

If lower temperatures and pressures than those described above are used, then Conversion takes longer than the times suggested above. The process can be used on dry powder or crystal forms of peptides or the peptides can be put into solution and then Converted. When peptides are put into aqueous solutions then pH becomes an important factor to monitor, adjust and control. In general, lower the pH solutions Convert faster than higher pH solutions and Conversion just about stops above pH 7.0.

Typical autoclave operating conditions suitable for the methods described herein are: steam heated to about 120° C. to 135° C. for about 15 minutes, or about 10 to 20 minutes, at a pressure of about 100 kPa or 15 psi, or about 10 to 20 psi, will be enough to make the Conversion in a reasonable period of time. One skilled in the art will be able to change and vary the conditions to monitor and control the rates of Conversion, by using measurements and assays as described herein.

The method of increasing peptide activity requires some heat over and above room temperature. Heat by itself or heat in the presence of steam and or heat in the presence of pressure can be used. The time it takes to convert depends on how much heat, and or steam and pressure and if relevant the acidity of the solution the peptides are in. Heat plus time is sufficient to make the make the changes or Conversion identified herein. How much time is required depends on how much heat is used and whether or not steam and pressure are used with the heat. Similarly, how much heat is required depends on how much time the peptides are heated and whether or not steam and pressure is used.

A few examples of possible heat options, with and without, steam; as well as various pressures that can be used to increase the toxicity of peptides are disclosed. One skilled in the art would be able to use these teachings and examples to determine many other possible temperatures, pressures, pH conditions and combinations thereof.

Examples of temperatures, times and pressures, with and without steam.
With steam: a) 110 C, 30 psi, 20 min.; b) 120 C, 15 psi, 15 min.; c) 130 C, 30 psi, 3 min., 8 min., 10 min. to 15 min. depending on container and whether covered or not.
Without steam (dry normal pressure): a) 120 C, 0 psi, 12 hrs,; b) 130 C, 0 psi, 6 hrs.; c) 140 C, 0 psi, 3 hrs.; d) 150 C, 0 psi, 2.5 hrs.; e) 160 C, 0 psi, 2 hrs.; f) 170 C, 0 psi, lhr.

It should be noted and understood that even moderate increases in temperature can effectuate the desired changes in the peptide, provided enough time is given for the reaction to proceed. For example, room temperature is typically in the range of about 20 to 25 C. When the temperature of the preparations is raised to as little as 40 C, the reaction can take place in a number of hours or days; however, the reaction at 40 C, with no steam and no pressure will proceed very slowly and could take as long as 2 years to complete. The reaction at 100 C, with no steam and no pressure could take as long as 6 months to complete. But if the reaction is run at 120 C, 15 psi, Conversion could be completed in 15 minutes.

The examples of heat, time, steam, and pressure, provided above can be used with wet or dry preparations. Dry preparation activity is important because in the commercial preparations of the peptide toxin, a dry preparation is easy to measure, transport, sell and use. The method of exposing dry powder to steam heat is especially preferred because the steam heat can also be used to disable and deactivate most living materials such as yeast hybrids that may be undesirable left over contaminates from the manufacture of the toxic peptides.

Another independent factor, in addition to heat, steam and pressure, that can be used to increase the activity of peptides is pH or acidity. Low pH, i.e. below 7, or acidity, can be used when the peptides are in solution and either at room temperature or in combination with the time, temperatures, pressure and steam factors discussed above.

Acidity and Acid conditions is believed to be an important factor that can influence the rate of Conversion. First it should be appreciated that the processes described above can take place when the peptides are in a dry form without water, but they can also be converted to their more active form when mixed with water, or when hydrated with sufficient water to form a measurable pH. Low pH or acid conditions, 7.0 or less has been found to be an independent factor that can be used to increase the rate and speed of Conversion. The optimal pH appears to be between about 1.5 and about 6, preferably between about 2 and about 5, more preferably between about 3 and about 4, more preferably about 3.5 but any acid conditions, 7.0 or lower, will increase the rate of reaction when the peptides are in solution. This is essentially an equilibrium reaction driven by pH. At a pH above 7.0 the reaction will be slow, the higher the pH the slower until it becomes so slow as to be essentially ineffective, when using aqueous reaction conditions. There will be some conversion at a pH slightly above pH 7.0 to about 7.5. At higher pH conditions the Conversion will be so slow as to effective and is generally considered to be of little commercial value.

In one embodiment the peptides are mixed with water, put in solution at a pH of 6.0 or less and Converted under steam and pressure at a temperature of between about 120° C. to about 150° C. for a rapid Conversion in less than about 10 minutes.

The Reaction. Without wishing to be bound by theory, and the procedures described do not require it, but to further advance the disclosure of the discovery, and to improve the teaching herein, we think the following reactions may take place during Conversion. When certain peptides are processed according the heat, pressure, steam, and acid regimes described herein they appear to lose the equivalent of a water molecule and so we sometimes call the process on of dehydration.

For purposes of illustration, we provide data for 2 sequences, SEQ ID NO: 119 (also called Hybrid+2) and SEQ ID NO: 121, both are provided in the examples and the sequence listing. These are two toxic peptides that differ only their N-terminal amino acids. SEQ ID NO: 119 has an N-terminal GS. SEQ ID NO: 121 does not have an N-terminal GS. SEQ ID NO: 121 has 39 amino acids and they are the same 39 C-terminal amino acids found in SEQ ID NO: 119. These toxic peptides are useful to demonstrate and explain Conversion.

We begin by explaining what Conversion is not. Conversion is not when a peptide with an N-terminal having an amino acid like glutamine, or Q, as in SEQ ID NO: 121, spontaneously forms a cyclic compound like pyroglutamic acid. For example the N-terminal glutamic acid of SEQ ID NO: 121 can form pyroglutamic acid. Here we call the spontaneous cyclization of either an N-terminal or internal amino acid having a free NH₃ group, the “NH₃ reaction.” The NH₃ reaction is not Conversion and it is not comparable to Conversion. We call Conversion the “2H+O reaction” or “H₂O reaction” or “dehydration reaction,” and it is completely different than the NH3 reaction. Both can occur with the same peptide as we prove in Example 5. The existence of two forms of a single peptide and the controlled ability to change one form into the other or at least the form having 2H+O into a form not having it is demonstrated with these two peptides and is characterized and explained below in the examples.

### Optimal Peptides for Conversion

We believe many peptides are suitable for Conversion, including those described in detail below. Toxic insect peptides or insect predator peptides have 2, 3 or 4 cystine bonds, which means they have 4, 6, or 8 cysteines. They are peptides of greater than about 10 amino acid residues and less than about 300 amino acid residues. More preferably they range in amino acid or aa size from about 20 aa to about 50 amino acids. They range in molecular weight from about 550 Da to about 350,000 Da. They show surprising stability when exposed to high heat and low pH. Toxic insect peptides have some type of insecticidal activity. Typically they show activity when injected into insects but most do not have significant activity when applied to an insect topically. The insecticidal activity of toxic insect peptides is measured in a variety of ways. Common methods of measurement are widely known to those skilled in the art. Such methods include, but are not limited to determination of median response doses (e.g., LD₅₀, PD₅₀, LC₅₀, ED₅₀) by fitting of dose-response plots based on scoring various parameters such as: paralysis, mortality, failure to gain weight, etc. Measurements can be made for cohorts of insects exposed to various doses of the insecticidal formulation in question. Analysis of the data can be made by creating curves defined by probit analysis and/or the Hill Equation, etc. In such cases, doses would be administered by hypodermic injection, by hyperbaric infusion, by presentation of the insecticidal formulation as part of a sample of food or bait, etc.

Toxic insect peptides are defined here as all peptides shown to be insecticidal upon delivery to insects either by hypodermic injection, hyperbaric infusion, or upon per os delivery to an insect (i.e., by ingestion as part of a sample of food presented to the insect). This class of peptides thus comprises, but is not limited to, many peptides produced naturally as components of the venoms of spiders, mites, scorpions, snakes, snails, etc. This class also comprises, but is not limited to, various peptides produced by plants (e.g., various lectins, ribosome inactivating proteins, and cystine proteases), and various peptides produced by entomopathogenic microbes (e.g. the Cryl/delta endotoxin family of proteins produced by various Bacillus species.)

The following documents are incorporated by reference in the US in their entirely, in other jurisdictions where allowed and they are of common knowledge given their publication. In addition they are incorporated by reference and known specifically for their sequence listings to the extent they describe peptide sequences. See the following:

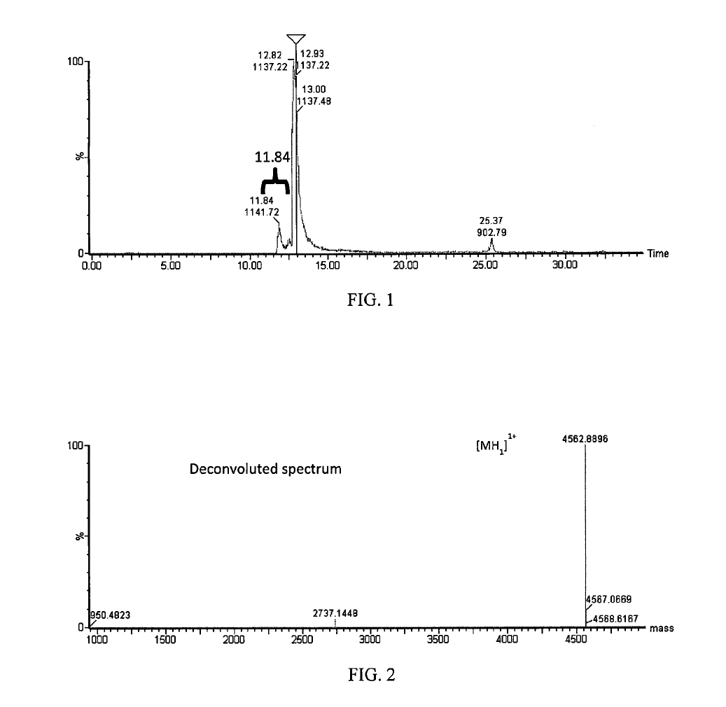

U.S. Pat. No. 7,354,993 B2, issued Apr. 8, 2008 specifically the peptide sequences listed in the sequence listing, and those numbered 1-39, and those named U-ACTX polypeptides, toxins that can form 2-4 intrachain disulphide bridges, and variants thereof, and the peptides appearing on columns 4-9 of the specification and in FIG. 1. EP patent 1 812 464 B 1, published and granted Aug. 10, 2008 Bulletin 2008/41, specifically the peptide sequences listed in the sequence listing, toxins that can form 2-4 intrachain disulphide bridges, and those as numbered 1-39, and those named U-ACTX polypeptides, and variants thereof, and the peptides appearing in paragraphs 0023 to 0055, and appearing in FIG. 1, of those patents.

Described and incorporated by reference to the peptides identified herein are homologous variants of sequences mentioned, have homology to such sequences or referred to herein which are also identified and claimed as suitable for Conversion according to the processes described herein including but not limited to all homologous sequences including homologous sequences having at least any of the following percent identities to any of the sequences disclosed her or to any sequence incorporated by reference: 30%, 35%, 40%, 45%, 50%, 55%, 60%, 65%, 70%, 75%, 80%, 85%, 90% or 95% or greater identity to any and all sequences identified in the patents noted above, and to any other sequence identified herein, including each and every sequence in the sequence listing of this application. When the term homologous or homology is used herein with a number such as 30% or greater then what is meant is percent identity or percent similarity between the two peptides. When homologous or homology is used without a numeric percent then it refers to two peptide sequences that are closely related in the evolutionary or developmental aspect in that they share common physical and functional aspects like topical toxicity and similar size within 100% greater length or 50% shorter length or peptide.

Described and incorporated by reference to the peptides identified herein that are derived from any source mentioned in the US and EP patent documents referred to above, including but not limited to the following: Toxins isolated from plants and insects, especially toxins from spiders, scorpions and plants that prey on or defend themselves from insects, such as, funnel web spiders and especially Australian funnel web spiders, including toxins found in, isolated from or derived from the genus Atrax or Hadronyche, including the genus species, Hadronyche versuta, or the Blue Mountain funnel web spider, Atrax robustus, Atrax formidabilis, Atrax infensus including toxins known as “atracotoxins,” “co-atracotoxins,” “kappa” atracotoxins, “omega” atracotoxins also known as ω-atracotoxin, U-ACTX polypetides, U-ACTX-Hv1a, rU-ACTX-Hv1a, rU-ACTX-Hv1b, or mutants or variants, especially peptides of any of these types and especially those less than about 200 amino acids but greater than about 10 amino acids, and especially peptides less than about 150 amino acids but greater than about 20 amino acids, especially peptides less than about 100 amino acids but greater than about 25 amino acids, especially peptides less than about 65 amino acids but greater than about 25 amino acids, especially peptides less than about 55 amino acids but greater than about 25 amino acids, especially peptides of about 37 or 39 or about 36 to 42 amino acids, especially peptides with less than about 55 amino acids but greater than about 25 amino acids, especially peptides with less than about 45 amino acids but greater than about 35 amino acids, especially peptides with less than about 115 amino acids but greater than about 75 amino acids, especially peptides with less than about 105 amino acids but greater than about 85 amino acids, especially peptides with less than about 100 amino acids but greater than about 90 amino acids, including peptide toxins of any of the lengths mentioned here that can form 2, 3 and or 4 or more intrachain disulphide bridges, including toxins that disrupt calcium channel currents, including toxins that disrupt potassium channel currents, especially insect calcium channels or hybrids thereof, especially toxins or variants thereof of any of these types, and any combination of any of the types of toxins described herein that have topical insecticidal activity, can be Converted by the processes described herein.

It should be understood that the same or other peptides can be conjugated to the peptides described herein. The conversion from Form 1 to Form is an internal conversion, the N and C terminal peptides are not affected and thus the N and C terminal amino acids can have covalent binding partners, be they long or short. We describe in detail binding partners that at up to 1000 amino acids in size, in addition to 900, 800, 700, 600, 500, 400, 300, 200, 100, 50 or fewer amino acids peptide conjugates are described.

Venomous peptides from the Australian Funnel Web Spider, genus Atrax and Hadronyche are particularly suitable and work well when treated by the methods, procedures or processes described by this invention. These spider peptides, like many other toxic peptides, including especially toxic scorpion and toxic plant peptides, become topically active or toxic when treated by the processes described by this invention. Examples of suitable peptides tested and with data are provided herein. In addition to the organisms mentioned above, the following species may also carry toxins suitable for Conversion by the process of this invention. The following species are named: Agelenopsis aperta, Androctonus australis Hector, Antrax formidabillis, Antrax infensus, Atrax robustus, Bacillus thuringiensis, Bothus martensii Karsch, Bothus occitanus tunetanus, Buthacus arenicola, Buthotus judaicus, Buthus occitanus mardochei, Centruroides noxius, Centruroides suffusus suffusus, Hadronyche infensa, Hadronyche versuta, Hadronyche versutus, Hololena curta, Hottentotta judaica, Leiurus quinquestriatus, Leiurus quinquestriatus hebraeus, Leiurus quinquestriatus quinquestriatus, Oldenlandia affinis, Scorpio maurus palmatus, Tityus serrulatus, Tityus zulianu. Any peptidic toxins from any of the genus listed above could be considered for Conversion according to the process in this invention.

The Examples in this specification are not intended to, and should not be used to limit the invention, they are provided only to illustrate the invention. [0067]As noted above, many peptides are suitable candidates for conversion. The sequences noted above, below and in the sequence listing are especially suitable peptides that can be converted. Some of these peptides have been Converted according to the procedures described herein as is described in the examples below.

```
SEQ ID NO: 60
(one letter code)
SPTCI PSGQP CPYNE NCCSQ SCTFK ENENG NTVKR CD
1 5 10 15 20 25 30 35 37
SEQ ID NO: 60
(three letter code)
Ser Pro Thr Cys Ile Pro Ser Gly Gln Pro Cys Pro Tyr Asn Glu Asn
  1 5 10 15
Cys Cys Ser Gln Ser Cys Thr Phe Lys Glu Asn Glu Asn Gly Asn Thr
                          20 25 30
Val Lys Arg Cys Asp
                  35 37
```

Named “ω-ACTX-Hv1a” it has disulfide bridges at positions: 4-18, 11-22 and 17-36. The molecular weight is 4096.

```
SEQ ID NO: 117
(one letter code)
GSSPT CIPSG QPCPY NENCC SQSCT FKENE NGNTV KRCD
1 5 10 15 20 25 30 35 39
SEQ ID NO: 117
(three letter code)
Gly Ser Ser Pro Thr Cys Ile Pro Ser Gly Gln Pro Cys Pro Tyr Asn
  1 5 10 15
Glu Asn Cys Cys Ser Gln Ser Cys Thr Phe Lys Glu Asn Glu Asn Gly
                          20 25 30
Asn Thr Val Lys Arg Cys Asp
                  35 39
```

Named “ω-ACTX-Hv1a+2” it has disulfide bridges at positions: 6-20, 13-24 and 19-38. The molecular weight is 4199.

```
SEQ ID NO: 118
(one letter code)
GSAIC TGADR PCAAC CPCCP GTSCK AESNG VSYCR KDEP
1 5 10 15 20 25 30 35 39
SEQ ID NO: 118
(three letter code)
Gly Ser Ala Ile Cys Thr Gly Ala Asp Arg Pro Cys Ala Ala Cys Cys
  1 5 10 15
Pro Cys Cys Pro Gly Thr Ser Cys Lys Ala Glu Ser Asn Gly Val Ser
                          20 25 30
Tyr Cys Arg Lys Asp Glu Pro
                    35 39
```

Named “rκ-ACTX-Hv1c” it has disulfide bridges at positions: 5-19, 12-24, 15-16, 18-34. The molecular weight is 3912.15

```
SEQ ID NO: 119
(one letter code)
GSQYC VPVDQ PCSLN TQPCC DDATC TQERN ENGHT VYYCR A
1 5 10 15 20 25 30 35 40 41
SEQ ID NO: 119
(three letter code)
Gly Ser Gln Tyr Cys Val Pro Val Asp Gln Pro Cys Ser Leu Asn Thr
  1 5 10 15
Gln Pro Cys Cys Asp Asp Ala Thr Cys Thr Gln Glu Arg Asn Glu Asn
                          20 25 30
Gly His Thr Val Tyr Tyr Cys Arg Ala
                  35 40 41
```

Named “rU-ACTX-Hv1a (”Hybrid“)+2” it has disulfide bridges at positions: 5-20, 12-25, 19-39. The molecular weight is 4570.51.

The examples below are intended to illustrate and provide further written description and support to this disclosure. They are not intended to limit the disclosure or the claims.

### EXAMPLES

### General Information about the Examples

SEQ ID NO: 119 is GSQYCVPVDQPCSLNTQPCCDDATCTQERNENGHTVYYCRA. SEQ ID NO: 119 has 41 amino acids. When properly folded, it has 3 disulfide bonds. It has the elemental composition of C₁₈₅H₂₇₆N₅₆O₆₈S₆. SEQ ID NO: 119 may be called the “+2 hybrid,” “Hybrid+2,” or the “plus 2 hybrid.”
SEQ ID NO: 121 is QYCVPVDQPCSLNTQPCCDDATCTQERNENGHTVYYCRA. SEQ ID NO: 121 has 39 amino acids and they are the same as the 39 “C” terminal amino acids in SEQ ID NO: 119. SEQ ID NO: 121 may be called, “native” or “native hybrid” OR “native hybrid peptide.”

The N-terminal amino acid of SEQ ID NO: 119 is “G,” glycine or Gly. The 2 N-terminal amino acids in SEQ ID NO: 119 are “GS” these amino acids are not part of the N-terminal of SEQ ID NO: 121. The N-terminal of SEQ ID NO: 121 is “Q” or glutamine.

It is possible for a sequence ending in glutamine, Q or Gln, like SEQ ID NO: 121 to spontaneously undergo cyclization, from glutamine to pyroglutamic acid. The reaction can be quick, can be spontaneous and does not require the addition of heat or acid. This amino group, sometimes N-terminal cyclization, is known to occur in peptides and it results in the peptide losing 17 mass units, atomic units or daltons, correlating with the 17 daltons of NH₃ lost upon the cyclization. We refer to this reaction as the “NH₃ reaction,” it is not what we call Conversion. We explain this reaction in more detail in Example 5, below.

We think a very different reaction occurs when heat is applied to a toxic peptide like SEQ ID NO: 121, and we refer to this reaction as Conversion or the “2H+O reaction.” Conversion results in a surprising increase in activity in the peptide which is an altogether different reaction, with the peptide having different properties as compared to what happens to a peptide that experiences the NH₃ reaction.

The increase in activity resulting from Conversion can be nearly 5 fold, or 5×, the data is shown below. When a toxic peptide is exposed to any of the Conversion conditions we describe herein, i.e. heat, pressure, steam, acid conditions for aqueous solutions, then we believe the “2H+O reaction” results in a peptide having increased activity.

The Conversion, or 2H+O reaction, results in a compound having one less water molecule than before the reaction starts. Below, we provide data showing that the peptide that results from Conversion results in a peptide with one less H₂O, or 18 dalton and is essentially dehydrated but is also much more robust and toxic a peptide, than the peptide was before it was Converted. That is the form of the peptide changes, such that the original form, herein called form 1 or Peak 1, has 18 more daltons as compared to the Converted Form 2 peptide.

Mass spec. evidence that shows the 2H+O reaction is not the same as the NH₃ reaction, rather, the 2H+O reaction results in the loss of 18 Daltons correlating with 18 Daltons of H₂O, rather than 17 Daltons of NH₃. We show the daltons of H₂O lost in the 2H+O reaction in Example 1; and the 17 Daltons of NH₃ lost is provided in Example 5.

### Example 1

Mass Spectrograph Peak 1/Form 1 and Peak 2/Form 2. In FIGS. 1-4, with captions and descriptions provided below, a mass spectrograph is shown of SEQ ID NO: 119 and it has 2 distinct peaks. The two peaks are identified with a large number in bold and a bracket shaped arrow pointing at a number. We refer to the two peaks as Peak 1 and Peak 2. The spectra in these figures was produced and analyzed using a Water/Micromass quadrupole time-of-flight (Q-Tof Premier) mass spectrometer on line with a Waters NanoAcquity UPLC system.

FIG. 1 shows a mass spectrum with an arrow showing Peak 1 is at 11.84.

FIG. 2 is a mass spec. of SEQ ID NO: 119, with a deconvoluted spectrum of Peak 1, shown in FIG. 1, where the deconvoluted Peak 1 of FIG. 1, has the value 4562.8896.

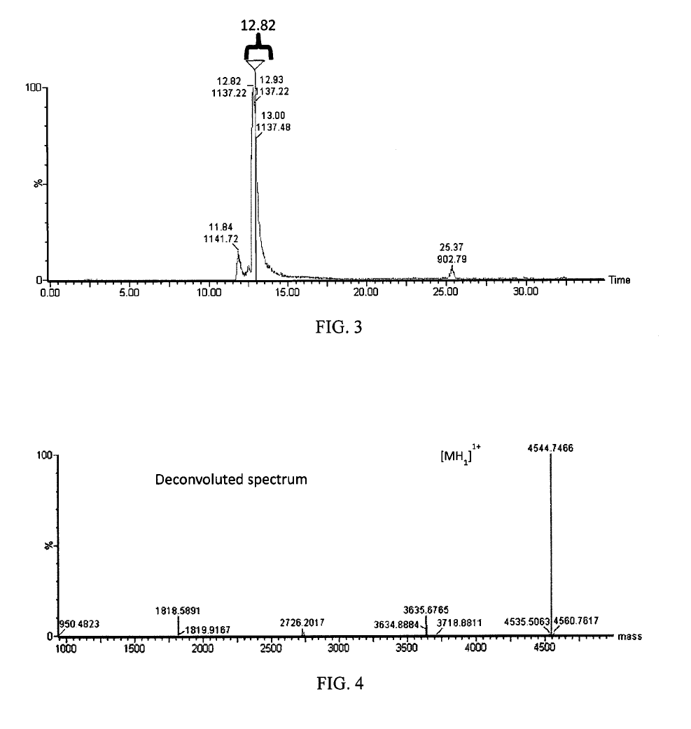

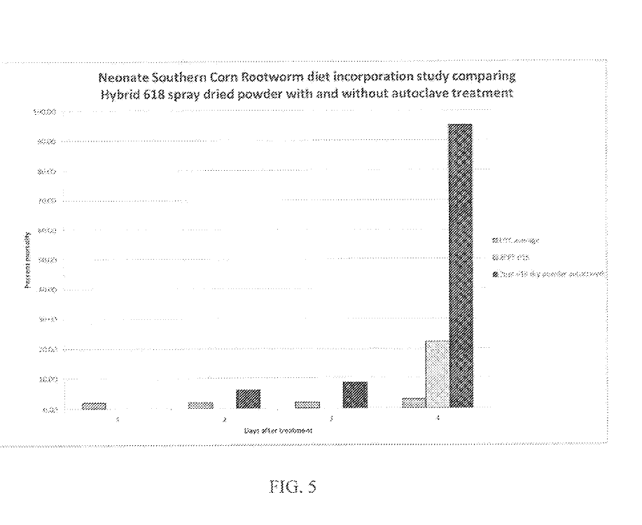

FIG. 3 shows a mass spectrum with arrow showing Peak 2 has the number 12.82. FIG. 4 is a mass spec. of SEQ ID NO: 119, with a deconvoluted spectrum of Peak 2 shown in FIG. 3, having the mass value 4544.8838.

FIGS. 1-4 show the difference between Peak 1 and Peak 2 is 18 Daltons or 2H+O.

When the two mass values from FIGS. 2 and 4 are subtracted from one another, 4562.8896-4544.8838=18.00, the value is 18 which corresponds to the mass value of a water molecule. Peak two is also referred to as the “dehydrated form” of the peptide, or the peptide lactone or as Form 2. Lactone is defined in the beginning of Part 2. Peak 2 indicated the peptide has taken the form that has lost a water molecule from its structure when compared to the structure that shows Peak 1.

The peptides and their forms, indicated by Peaks 1 and 2, were isolated and their activity compared. The examples below provide comparisons of the activity of the original form, called any of the following: Peak 1, Form 1, native, acid form, peptide acid, original, preConverted, unconverted, or not Converted form of the peptide. Form 1 is the form or acid form that heated or acidified in order to turn it into Form 2 or the lactone form or peptide lactone as lactone is defined in Part 2. In some of these examples the heat treatment is an autoclave treatment, at about 121° C. for 20 minutes at 21 psi., or if the peptide is in liquid form it means lowering the pH to under 7.0 in order to Convert the peptide to what is called any of the following: Peak 2, Form 2, the lactone form, the peptide lactone, (as lactone is defined in Part 2.) the dehydrated form of the peptide, or the Converted form of the peptide.

### Example 2

Diet Incorporation Study. The graph in FIG. 5, shows a comparison of the toxicity of the peptide of the original form, Peak 1, peptide acid, unConverted, compared to the toxicity of the peptide of the lactone form, or peptide lactone, after treatment or Converted, indicated by Peak 2. Both forms are also compared to a control. FIG. 5. Shows the percent of dead larvae, (100% would be all 16 larva dead) on days 1, 2, 3, and 4 days after the hungry catapillers were fed either control or treated diets. A peptides used in this study was SEQ ID NO. 119 and they were formulated into a spray dried powder called either powder 618 or 618 hybrid powder, both terms mean the same thing. The insects were dosed at the rate of an equivalent dose of 2 ppt (parts per thousand) in their feed. Peak 1 is the original peptide before Conversion or treatment, this is also called “traditional 618” or simply 618 powder or dry powder. Peak 2 is the peptide after Conversion or treatment, in this case after autoclaving for 20 minutes at 121° C. and 21 psi. i.e. high temperature, steam and pressure. The Peak 2 is named “6-18 dry powder autoclaved” in FIG. 5. FIG. 5 provides data in bar graph form for three sets of data or bars over each number in the horizontal or X axis, the number being the number of days following feeding the insects used in the study, called a southern corn rootworm (SCR) which is actually an insect, the test was performed on the larva stage. Sixteen insect larvae were used to begin each trial. The legend is shown in FIG. 5, it explains the large dark bar seen above day 4 to the right of the three grouped bars above day 4, is the result of feeding Form 2, the peptide lactone, to the insects. Peak 2, of the mass spec. is the Converted Form 2, the peptide lactone form, of the peptide. In FIG. 5, at day 4, Peak 2, the dark bar shows the mortality from the larvae ingesting Converted Form 2 of the peptide. In this case Form 2 was Converted by autoclaving Form 1. At day 4, there is a 95% level of rootworm mortality from Form 2, the peptide lactone, compared to about 22% mortality for Form 1, the peptide acid, or the form of the native or unconverted peptide. The control on day 4 has less than 5 percent mortality. The caterpillars were fed either untreated insect diet, i.e. a control, this is the first bar of each day, a fine grey cross hatch in FIG. 5. The second bar, with a larger black and white cross hatch pattern, shows the data for the caterpillars that were fed the peptide of Form 1, indicated by Peak 1 of the mass spec., this is the peptide before Conversion. The third bar, with a find dark bar shows the data for the caterpillars that were fed Form 2, indicated by Peak 2 in mass spec. analysis. Days 1-4 after feeding are shown with most of the mortality occurring on day 4. The Y-axis shows the percent of larvae that are dead, and there were 16 live larvae used at the start.

Four days after the insects were fed, the difference in the percent mortality between the traditional 618 (before Conversion) and the autoclaved 618 (after Conversion) becomes pronounced. The autoclave treatment lead to a quicker speedier death following the caterpillars eating treated food. The number of insects dying that were treated with Form 2, is about 95%, compared to the 618 dry powder Form 1, native peptide or peptide not Converted, Form 1, is about 22%. Autoclaving the normal peptide did not deactivate the hybrid protein as expected, instead, it improved its activity. There could be several reasons for the dramatic change in potency.

Methods: Insects: SCR are purchased from Crop Characteristics (Farmington, Minn.). Insects were received as “ready to hatch” on filter paper. The insects were hatched at room temperature (26 C) and left in the plastic bag they were shipped in. The insects were hatched after 1-2 days and were used the day of hatch for the assay.
Media: SCR larval diet was purchased from Bioserve (Product# F9800B, Frenchtown, N.J.). To make 100 mL of diet, 100 mL of DI water is boiled with 1.44 g of the provided agar. Solution is boiled until the agar is fully dissolved. Then 13.28 g of diet and 460 ul of KOH are added and media is mixed on warm stir plate until homogeneous. Media is then aliquoted into 20 mL portions and cooled to 65 C in a water bath.

Treatments: The 618 treatments were prepared using the calculation of 25% AI. A 10 ppt solution was made (10 mg/mL) by mixing 260 mg of powder with 6.5 mL of water. The solution is mixed thoroughly and sonicated if necessary to dissolve all the powder completely. 200 mg of 618 powder was put in a glass jar with a screw on top. The powder was then autoclaved on the 20 minute Dry cycle with the cap loosened. After the autoclave cycle, the powder had absorbed some liquid. 5 ml of water was then added to the powder and mixed well to dissolve. 5 mL of either water or treatment is then added to the 20 mL of 65 C food and mixed well and 1 mL of DI is then transferred to each well of the bug condos (Bioserve Product# BAW 128) using a repeat pipetter and allowed to cool.

Insects are then applied once media has cooled and set (20 min), one per well, using a paint brush to transfer SCR. Wells are then sealed with perforated lids (Bioserve Product# BACV16) and left on the light cart in the insect lab.

### Example 3

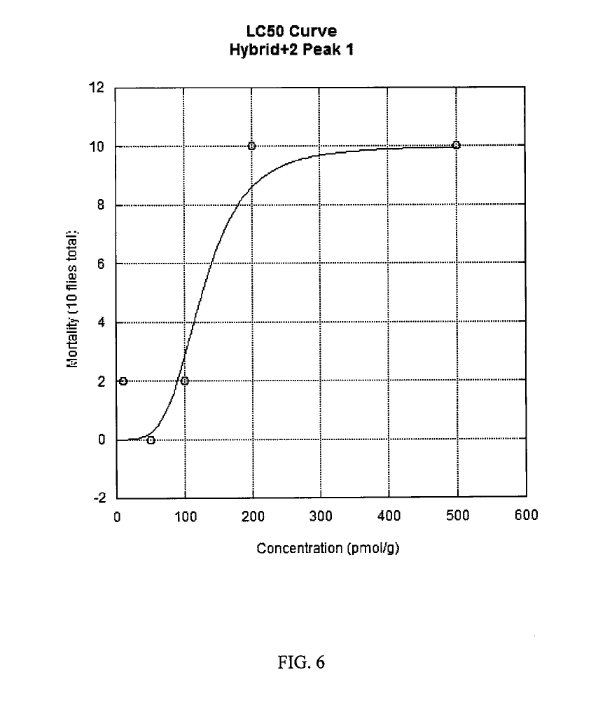

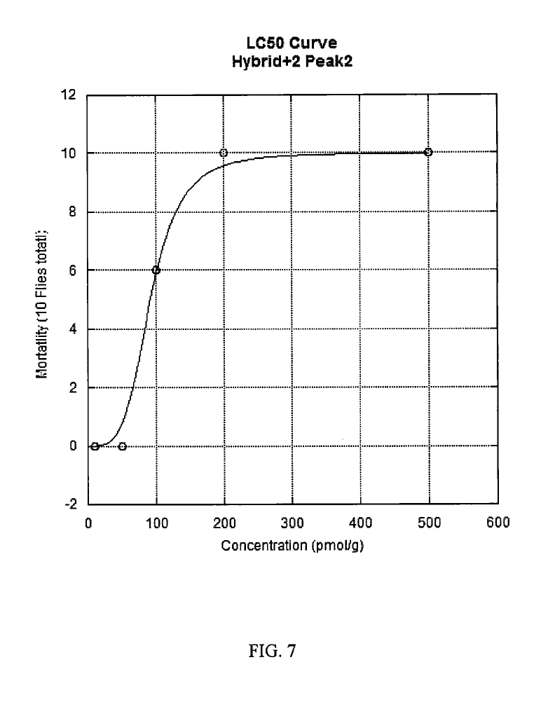

Bioassay comparison. Results of a bioassay comparison are shown in FIGS. 6 and 7. Peak 1 and Peak 2 were separately prepared and separately isolated from liquid chromatography columns, similar to those used to produce the studies shown in FIGS. 1-4. The peptide of SEQ ID NO: 119 was used for this comparison. Either Peak 1, the pre Conversion peak, or Peak 2, the after Conversion peak, was taken and made into a measured concentrate that was then administered by injection into houseflies. The LD₅₀ or lethal dose of 50% of the flies was determined as a concentration of μmols/gram. The flies weighed from 12 to 20 mg. There were 10 flies in each sample. Differences in molecular weight between Peak 1 form and Peak 2 form were not considered when preparing the standard μmol/g solutions. All the solutions that made the LD 50 solutions were made from what we call “Super LC” or Super Liquid Concentrate, using RpHPLC, or Reverse phase High Pressure Liquid Chromotography.

FIG. 6. Is a bioassay comparison of Peak 1 and Peak 2 where each peak fraction was separately prepared from liquid chromatography. The Peak 1 bioassay results are shown.

FIG. 7. Is a bioassay comparison of Peak 1 and Peak 2, where each peak fraction was separately prepared from liquid chromatography. Peak 2 results are shown.

The results of the Bioassay comparisons as lethal dose 50 are provided in Table 1, below.

Table 1, below.

| Solution | LD50 (pmol/g) |
|---|---|
| Hybrid + 2 Peak 1 | 127 |
| Hybrid + 2 Peak 2 | 92 |

### Example 4

Stability pH Study. This was both a stability and a pH study. It compares pre Conversion or Form 1, to post Conversion or From 2 peptides. The study used the peptide of SEQ ID NO 119 and shows that, in addition to heat, a decrease in pH, that is the lowering of the pH of a solution of peptide, with acid or any means to lower the pH to make it 7.0 or below, will result in increased Conversion of the peptide from Form 1 to Form 2.

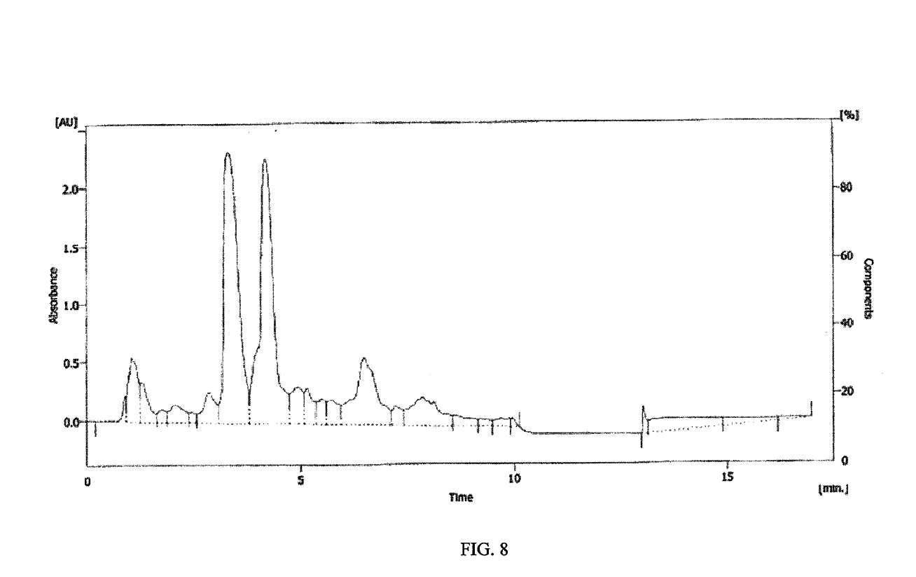

The Stability pH Study results are shown in FIGS. 8-10.

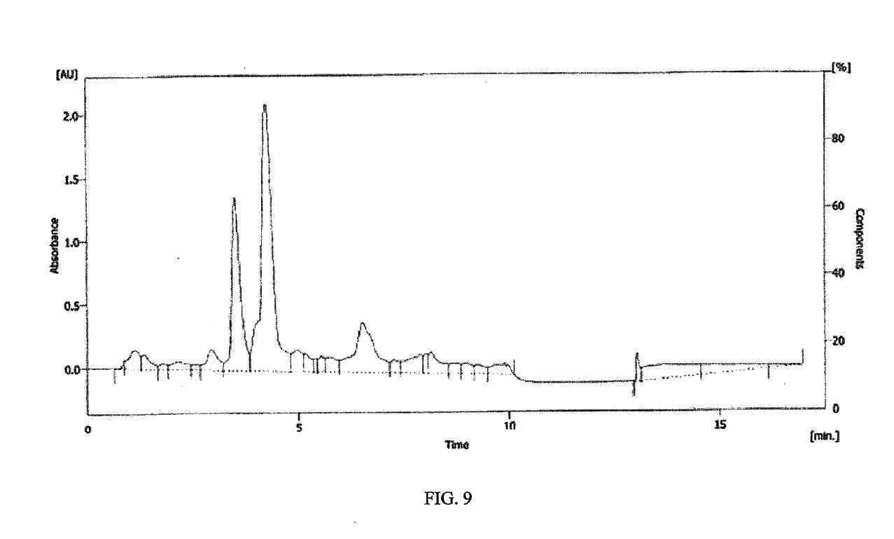

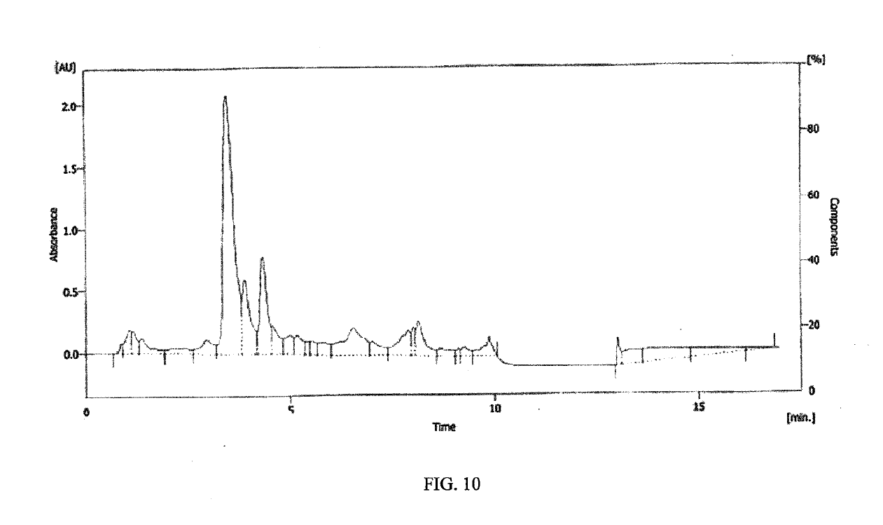

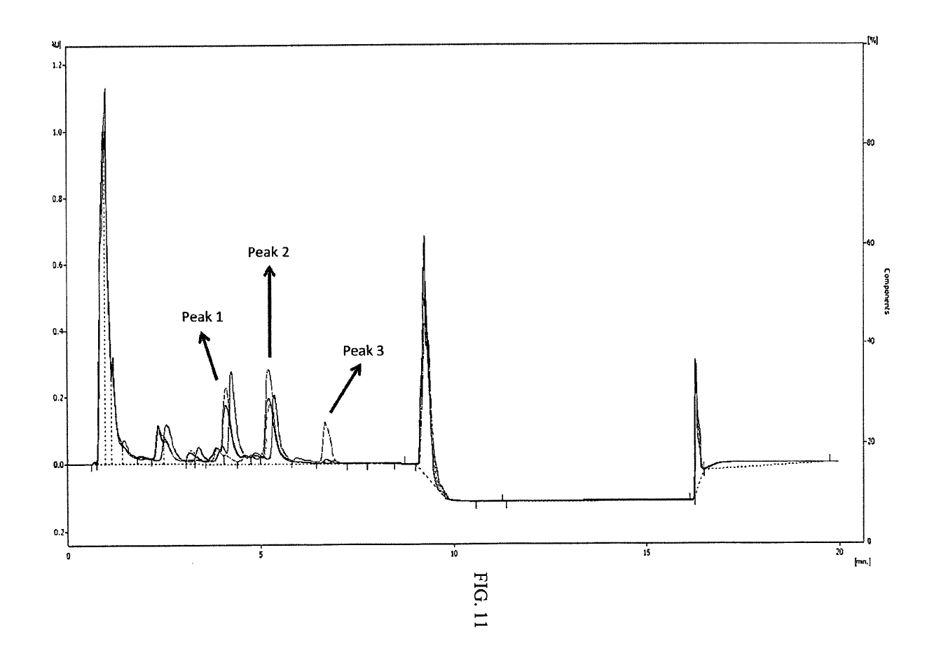

FIG. 8 is a Mass Spec of SEQ ID NO: 119 at pH 5.6. FIG. 9 is a Mass Spec of SEQ ID NO: 119 at pH 3.9. FIG. 10 is a Mass Spec of SEQ ID NO: 119 at pH 8.3. FIGS. 8, 9 and 10 show, but do not specifically identify, Peak 1 and Peak 2. In all three figures Peak 1 is to the left of Peak 2, and both are the larger Peaks in the figures. These three figures, FIGS. 8, 9 and 10 are representative of the mass spec. results produced in this study. The data from these figures and other data is presented in Tables 2-7, below. Peak 1 elutes before Peak 2. In FIG. 8, the two peak heights are about the same. In FIG. 9, Peak 2 is greater than Peak 1. In FIG. 10, Peak 1 is greater than Peak 2. All the samples in this study were prepared by adding 2 mL pH 2 or pH 10 buffer to 2 mL Super Liquid Concentrate (54PPT). Samples were analyzed on Agilent HPLC. A 5 microliter injection volume was used. Results are described below.

Table 2, below.

Sample 1 at pH 3.9 and 8.3

| Sample | Peak 1 Height | Peak 2 Height |
|---|---|---|
| LC pH 5.6 | 2360 | 2225 |
| LC pH 3.9 | 2948 | 1630 |
| LC pH 8.3 | 2000 | 1526 |

Observation. Slight decrease in peak 2 height in both pH solutions.

Table 3, below.

Sample 2 at 25° C. for 24 hrs at pH 3.9 and 8.3

| Sample | Peak 1 Height | Peak 2 Height |
|---|---|---|
| LC pH 5.6 | 2359 | 2215 |
| LC pH 3.9 | 2001 | 1670 |
| LC pH 8.3 | 2023 | 1527 |

Observation. Slight decrease in peak 2 height in both pH solutions

Table 4, below.

Sample 3 at 25° C. for 96 hrs at pH 4.0 and 7.8

| Sample | Peak 1 Height | Peak 2 Height |
|---|---|---|
| LC pH 5.6 | 2341 | 2198 |
| LC pH 4.0 | 1887 | 1795 |
| LC pH 7.8 | 2038 | 1355 |

Observation. Larger decrease in peak 2 height in higher pH solution

Table 5, below.

Sample 4 at 40° C. for 72 hrs at pH 3.9 and 8.3

| Sample | Peak 1 Height | Peak 2 Height |
|---|---|---|
| LC pH 5.6 | 2325 | 2275 |
| LC pH 3.9 | 1365 | 2104 |
| LC pH 8.3 | 2082 | 782 |

Observation. Decrease in peak 1 height in lower pH solution. Decrease in peak 2 in higher pH solution.

Table 6, below.

Sample 5 at 75° C. for 1 hr. at pH 3.9 and 8.3

| Sample | Peak 1 Height | Peak 2 Height |
|---|---|---|
| LC pH 5.6 | 2359 | 2272 |
| LC pH 3.9 | 1807 | 1869 |
| LC pH 8.3 | 2008 | 1493 |

Observation. Slight decrease in peak 2 height in higher pH solutions

Table 7, below.

Sample 6 at 75° C. for 3 hr at pH 3.9 and 8.3

| Sample | Peak 1 Height | Peak 2 Height |
|---|---|---|
| LC pH 5.6 | 2117 | 2111 |
| LC pH 1.6 | 855 | 800 |
| LC pH 3.9* | 1038 | 1716 |
| LC pH 7.5 | 1630 | 798 |
| LC pH 9.4* | 689 | — |

*1:3 Dilution with buffer to get proper pH

Observation. Decrease in peak 1 height in pH 3.9 solution. Decrease in peak 2 height in pH 7.5 solution. Loss of peak 2 in pH 9.4 solution.

This study shows Peak 1, the pre Conversion peptide Form 1 and Peak 2, the Converted peptide Form 2, after they are taken up in aqueous solution and adjusted to different pH or acidity levels. The study reveals that in solution it is difficult and there is little or no natural movement from Form 1 to Form 2. The peptide form does not convert unless the pH is lowered to 7.0 or less, preferably 6.0 or less, more preferably 5.0, 4.5, 4,0, 3.5, 3.0. 2.5, 2.0 or less, 3.2 to 3.5 to 3.8 and all pH values between 3 and 4 are preferred. This study also reveals that the lower the pH, the quicker Form 1 will convert to Form 2. Form 2 is the dehydrated or less 2H+0, or less 18 dalton form of the peptide.

### Example 5

Non Converting isoforms. We have shown that SEQ ID NO: 119 can form an isoform with the loss of 18 Daltons in M.W. at higher temperature. In Example 2 we showed close to a 5 fold increase in insecticidal potency when the original form of SEQ ID NO: 119, Form 1, as a powder, was autoclaved to make it Convert to Form 2 and then it was tested by adding to the diet of the Southern Corn Rootworm, larva set. However, this transformation in SEQ ID NO: 119, a hybrid peptide, has not been noticed in a peptide like SEQ ID NO: 121, a native peptide. In contrast to SEQ ID NO: 119, in SEQ ID NO: 121 there is a an N-terminal Gln, which may cyclized itself to N-Pyr with loss of a NH₃, i.e. loss of 17 daltons in M.W. These two chemical modifications, loss of H₂O and loss of NH₃, are difficult to differentiate because the loss of M.W. in these two processes is so close. We used analytical HPLC and sensitive TOF LC/MS methods to evaluate whether both of these chemical modifications can happen to a sequence like SEQ ID NO: 121, which we also call the native hybrid peptide. The data below shows the Conversion can be induced in a native hybrid peptide when it is subjected to appropriate conditions as we describe herein for this process.

Materials and Methods. SEQ ID NO: 121 was made from Hybrid-ACTX-Hv1a K. lactis strain, pLB12D-YCT-24-1. Agilent HPLC system with Onyx 100 monolithic C18 HPLC column was used to analyze the SEQ ID NO: 121 peptide production and isoform formation.

The LC-MS system is located at Launch MI Lab in SMIC, and consists of a Waters/Micromass quadrupole time-of-flight (Q-Tof Premier) mass spectrometer on-line with a Waters NanoAcquity UPLC system. Sample was diluted 1:50 in 0.1% formic acid in water.

Method A.

Five μL of sample was injected onto a Waters BEH130 C-18 Symmetry column (0.3 mm ID×15 cm) at a flow rate of 5 uL/min. Reverse-phase separation was achieved using a linear gradient from 0.1% mobile phase B (water with 0.1% formic acid) to 40% mobile phase B (100% acetonitrile with 0.1% formic acid) over 25 minutes, 85% B at 25.5 minutes, 85% B at 27.5 minutes, and 0.1% B at 28 minutes.

Method B.

Ten to 30 uL μL of sample was injected onto a Waters C-18 X-Bridge Column (4.6 mm ID×50 mm) at a flow rate of lmL/min. Reverse-phase separation was achieved over 15 minutes using a linear gradient of 99% mobile phase A (water with 0.1% formic acid) to 95% mobile phase B (100% acetonitrile with 0.1% formic acid) over 6 minutes, 95% B at 11 minutes, and 1% B at 11.2 minutes for a total run time of 18 minutes.

Column effluent was sampled by the mass spectrometer via an electrospray ionization source. Waters Masslynx 4.1 software was used for instrument control and MS and MS/MS data acquisition. Within Masslynx the MaxEnt 3 algorithm was used for deconvolution of multiply charged ions to a calculated monoisotopic M+H mass valu

Method C.

The LC-MS system consisted of a Waters/Micromass ZQ spectrometer with an electrospray ionization source. The sample was injected onto a Zorbax SB-C18 column (2.1×30 mm) at a flow rate of 1 mL/min. Reverse-phase separation was achieved over 3.1 minutes using a linear gradient of 96% mobile phase A (water with 0.1% formic acid) to 98% mobile phase B (100% acetonitrile with 0.07% formic acid) using a diode array detector (210 to 300 nm).

Results and Discussion. Production of SEQ ID NO: 121, aka native hybrid peptide, production strain, pLB24-YCT-24-1, was cultured in Defined Medium with 2% sorbitol as carbon source at 23.5 C for 6 days. The OD600 reached 30 at the time when the condition medium was collected after removal of cells. 300 μl of the conditioned medium was injected into Agilent HPLC analytic system and a yield of native hybrid peptide was determined as 164 mg/L.

Agilent HPLC evaluation of native hybrid isoforms. The collected native hybrid conditioned medium was treated at 4 C, room temperature (˜23 C) and 50 C for 24 hours before analysis by Agilent analytic HPLC with loading of 300 μL at of each. The HPLC chromatographs of native hybrid peptide samples treated at different temperature are shown in FIG. 11. Three HPLC peaks, of which UV absorbance changed with temperature, have been identified at retention time of 4.2 min, 5.4 min and 6.9 min. We believe Peak 1 is the least hydrophobic isoform and that Peak 3 is the most hydrophobic isoform.

Peak 1, indicating Form 1, was the most abundant isoform initially, but Peak 1/Form 1 can transformed into isoforms Peak 2 and Peak 3 with time and higher temperature. We demonstrate that a 50° C. treatment for 24 hr. will almost make Peak 1 disappear (to only 5.6%). Conversely, Peak 2 and Peak 3 isoforms increase with temperature and increase faster with higher temperature.

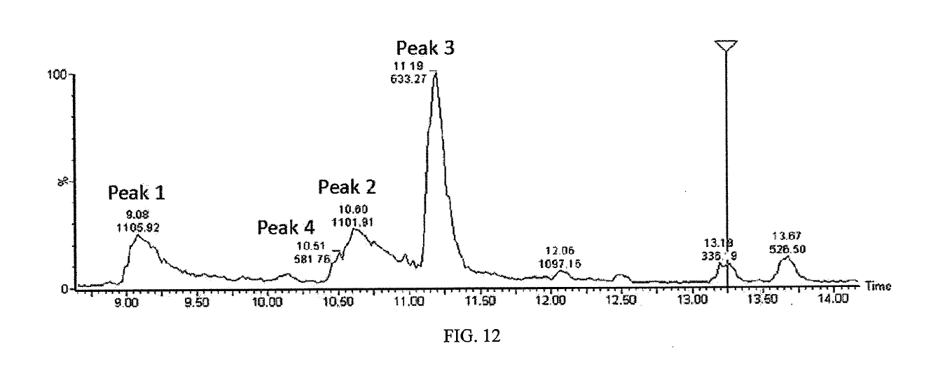

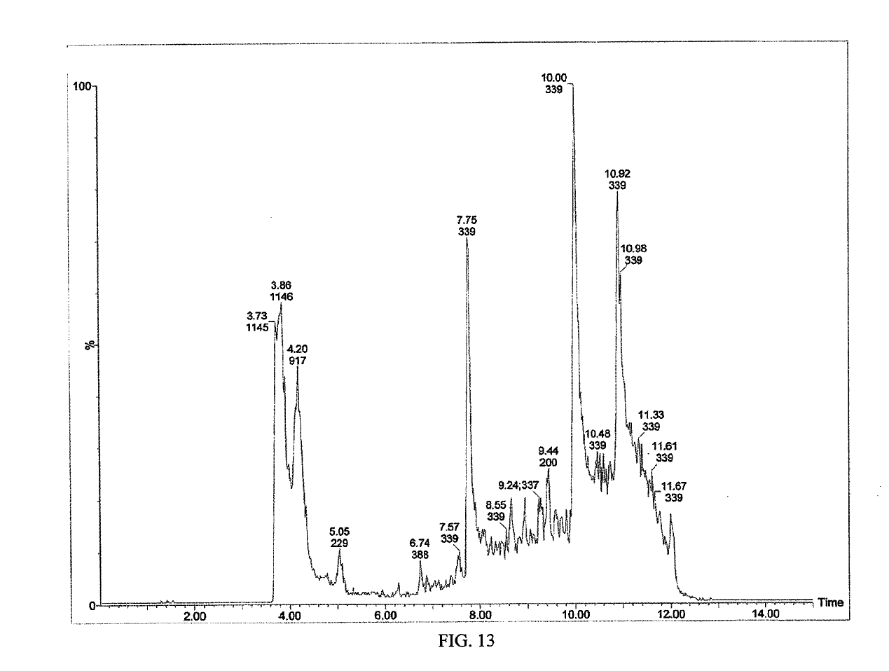

FIG. 12 shows the results of a TOF MS Evaluation (Time Of Flight Mass Spec.) of the isoforms of the native hybrid peptide. The results are presented in the form of a Base Peak Intensity (BPI) chromatograph. In order to identify Peak 1, Peak 2 and Peak 3 in FIG. 11, a time-of-flight Mass Spectrometry was performed using the native peptide conditioned medium with RT treatment. The time-of-flight MS can isolate the isotopic m/z ratio generated from the MS instruments, therefore this MS method can detect the monoisotopic M.W. of the peptide. The theoretical monoisotopic M.W. of native hybrid is 4417.812. The TOF MS detected 4 isoforms of native hybrid peptide in the conditioned medium sample.

One isoform detected by TOF MS was the one with M.W. of 4417.6826, which represents the “native” native hybrid peptide, i.e. unmodified native hybrid, it is labeled as Peak 1 in FIG. 11 and Peak 1 in FIG. 12.

A second isoform detected had a M.W of 4399.6455. This isoform has 18 dalton loss in M.W. from the “native” isoform, indicating loss of a water molecule. This isoform, with a loss of H₂O, is not labeled in FIG. 11 and labeled as Peak 4 in FIG. 12.

A third isoform detected had a M.W. of 4400.6660. This isoform had 17 dalton loss in M.W. from the “native” isoform and likely a loss of NH₃. This isoform with a loss of NH₃ is labeled as Peak 2 in FIG. 11 and is labeled as Peak 2 in FIG. 12, From a previous study of TEP fusion hybrid+2, the N-Gln peptide will naturally cyclize to N-pyroglutamic acid with loss of a NH₃. Therefore, the third isoform represents the peptide with N-Gln cyclized to N-Pyr, since native hybrid peptide has a N-Gln and this is shown as Peak 2 in FIG. 12.

A fourth isoform is the combination of loss of both a H₂O and a NH₃ molecule, resulting in an isoform with M.W. of 4382.6313. The isoform with a loss of both H₂O and NH₃ is labeled as Peak 3 in FIG. 11, and Peak 3 in FIG. 12.

These results show there are at least two chemical modifications possible in the native hybrid peptide molecule, both N-terminal glutamine cyclization to pyroglutamic acid, and a dehydration reaction. From the TOF MS based peak intensity chromatograph, the isoform with only a H₂O loss was barely detectable. This is consistent with the HPLC evaluation in which only 3 peaks have been detected. Loss of a H₂O molecule can make the peptide more hydrophobic and further loss of a NH₃ can make the peptide even more hydrophobic. We can predict that loss of H₂O will shift the native hybrid peak to a later retention time in HPLC chromatograph. Loss of both H₂O and NH₃ will further shift the peak to an even later retention time.

### Part 2

In Part 1 we describe how it is possible to artificially manipulate a toxic peptide with mechanical or chemical means such as temperature, pressure, strong and/or weak acids, in order to transform a peptide from its native state or what we call Form 1 into the useful state we call Form 2. The Form 2 composition may be referred to herein as the “carbonyl”, “activated carbonyl”, “lactone”, “lactone like”, and/or “lactone like form.” In this document we usually refer to this From 2 composition simply as a lactone or peptide lactone. The structure of these compounds has no dictionary definition in this document, here they are defined by the characteristics we describe here. Here a “lactone” has the properties we attribute to the Form 2 compound. We use the word “lactone” and peptide “lactone” because these compounds react like a lactone. We describe how to make them, how to identify them, how to isolate them and how to use them. We provide data to show these peptide lactones are more biologically active than the native peptides and that they are very useful and versatile. They are stable intermediates that can be used to make other valuable compounds. In Part 2 we show how the peptide lactone can be made into two different and stable active compounds, useful as stable intermediates to make a variety of other compounds.

In Part 2 we describe peptide hydrazides, peptide hydrazones and we teach how to make and use them. A hydrazide or peptide hydrazide results from the reaction of the Part I peptide lactone with hydrazine. The other stable intermediate compound we describe we call a hydrazone or peptide hydrazone. A peptide hydrazone results from the reaction of a peptide hydrazide with a carbonyl compound. What is especially useful about peptide hydrazones is that they can be covalently bonded with other useful moieties such as alkyl chains and or pegylated products and then used for a variety of purposes, some of which we describe here. The ability to create an alkylated protein, in the manner we describe, is very useful. The ability to easily produce a pegylated protein, in the manner we describe is, perhaps, even more useful. Pegylated proteins have been used to reduce the immunogenicity of proteins, to decrease the metabolism of proteins and to increase the bioavailability of proteins. We believe our techniques, disclosed here for the first time, can be used to create pegylated proteins with exceptional value. These techniques can be used to make alkylated and pegylated proteins, and other types of proteins, more easily, quicker and at a lower cost than previously possible. One protein enhanced by pegylation is insulin.

We are able to demonstrate that the peptide lactone, the peptide hydrazide, and the peptide hydrazone, these can be either “peptide intermediates,” novel, chemically stable, chemically useful compounds used to react with other compounds, like PEG4Ketone (VIII) in Example 11 and they can be final products like the pegylated peptides or pegylated peptide hydrazones in Example 11 showing a novel pegylated toxic peptide hydrazone having greater activity than would a similar toxic peptide having no pegylation. The peptide lactone and peptide hydrazide provide a single discrete site on these peptides or peptide acids where functional groups are added. The peptide and toxic peptide products and intermediates provide a single discrete chemical handle with unique chemistry synthetic or biological molecules more useful and functional. For example, this chemistry allows one to mono functionalize with a pegylation chain at a single site of the polypeptide. Another example is that it could allow one to mono attach molecules at one discrete site on the peptide or peptide acid such as a periodated digested glycosylated peptide or other carbohydrate. These peptide intermediates can be used to produce a wide range of products. We show that these toxic peptide intermediates are useful with good activity and provide more reaction options than the typical toxic peptide. We understand that pegylated toxic petides are even more active than unpegylated peptides.

PEGylation or pegylation is the linking of a peptide to polyethylene glycol and/or polypropropylene glycol or (PEG). Once linked to a peptide, each PEG subunit becomes tightly associated with two or three water molecules, which have the dual function of rendering the peptide more soluble in water and making its molecular structure larger. In a first generation protein pegylation the PEG attaches to one or more of several potential sites on the protein, such as to lysine and N-terminal amines. A problem with this approach is that a population of modified peptides can contain a mixture of molecules with PEG attached to different lysines, as well as molecules with different numbers of linked PEGs. This variability in modification diminishes the purity of the finished product and impedes reproducibility.

There have basically been two other more modern approaches used to add PEG to proteins in a more controlled manner; either A) alter the PEG to make it more reactive or B) alter the protein to provide special sites for PEG attachment.

A PEG method type A), alter the PEG, is described in U.S. Pat. No. 4,179,337, Davis et al., issued Dec. 18, 1979, incorporated herein by reference, specifically as to its descriptions of polymers suitable for pegylation. This patent describes modifying the polymer at one end either by the alteration of the terminal group or by the addition of a coupling group having activity to the peptide and reacting the activated polymer with the peptide. This method was used to pegylate insulin and other hormones. See U.S. Pat. No. 4,179,337.

A PEG method of type B), modify the peptide rather than the PEG, is to add a cysteine where desired to generate site-specific PEGylation at places chosen to minimize interference with the peptide's biological function, while decreasing the peptide's immunogenicity. PEG-maleimide, PEG-vinylsulfone, PEG-iodoacetamide, and PEG orthopyridyl disulfide are thiol reactive PEGs that have been created to PEGylate free cysteine residues. This approach has been used in a number of ways including making monoPEGylated human growth hormone analog. See Peptide PEGylation: The Next Generation, by Baosheng Liu, Pharmaceutical Technology,Volume 35.

The process described herein is a new and different method compared to anything used before and it allows for specific attachment of the PEG to a specific site on the protein. The novel method we describe provides for PEG attachment to the peptide using a PEG carbonyl reaction to a peptide hydrazide and is described in detail below. It can be used with any linear or branch polymer having a molecular weight of between about 500 to about 20,000 daltons selected from the group consisting of polyethylene glycol and polypropropylene glycol. The polymer may be unsubstituted or substituted by alkoxy or alkyl groups where the substituting groups possessing less than 5 carbon atoms. The benefits of being able to make a PEG toxic insect peptide are substantial and described above in the Summary of Invention.

General Reactions.

I)The Peptide Hydrazide.

The peptide hydrazide is made from the peptide lactone (see Part 1) and hydrazine to form a peptide hydrazide. The peptide hydrazide is essentially made in a three step procedure. The peptide lactone is mixed with hydrazine monohydrate. The mixture is stirred to solution to to form the peptide hydrazide, and the peptide hydrazide is purified.

One of ordinary skill in the art would be able to produce many versions of this procedure, for example, the mixture of peptide lactone and hydrazine should be stirred well to form a solution. The peptide hydrazine formed in this solution can then be purified by a variety of methods, such as by prepative HPLC.

We teach both mixing the aqueous solution of the peptide lactone and the solution of hydrazine added as hydrazine monohydrate and then stirring well at room temperature. The peptide hydrazide can then be purified. We used HPLC for purification, other options, known to one skilled in the art are available. Procedures of this type are well known to the ordinary chemist and the procedures outlined herein may be varied considerable by one skilled in the art. Other options for collection and purification could be used. In the examples below both relatively pure and impure samples of lactone were used as the starting material and both of these resulted in high purity Peptide Hydrazide (I) and are described in Example 6. Other procedures could be used. In Example 6a and 6(b) the peptide lactone and hydrazide are mixed together and stirred. Purification steps can vary widely and various options are available and known to one skilled in the art.

II) The Peptide Hydrazone.

The peptide hydrazone and peptide hydrazide are important intermediates. Different types of peptide hydrazones can be made depending on what functional groups are desired for the peptide. Here we show various examples of different peptide hydrazones. Examples of hydrazones are shown in Examples 8-11. One skilled in the art will understand these are but representative and illustrative not limiting examples, other reagents and conditions could be used.

These examples use the peptide hydrazide with one or another type of carbonyl to create novel peptide hydrazones like the examples of Formula (II), (III), (VI) and (IX). Some examples of reactive carbonyl compounds producing novel pegylated proteins are provided.

Hexanal is added to a hydrazide to produce Hydrazone (II).

The reactions discussed above are shown in the structures below with details provided in the descriptions below and supporting data can be found in FIGS. 13-26.

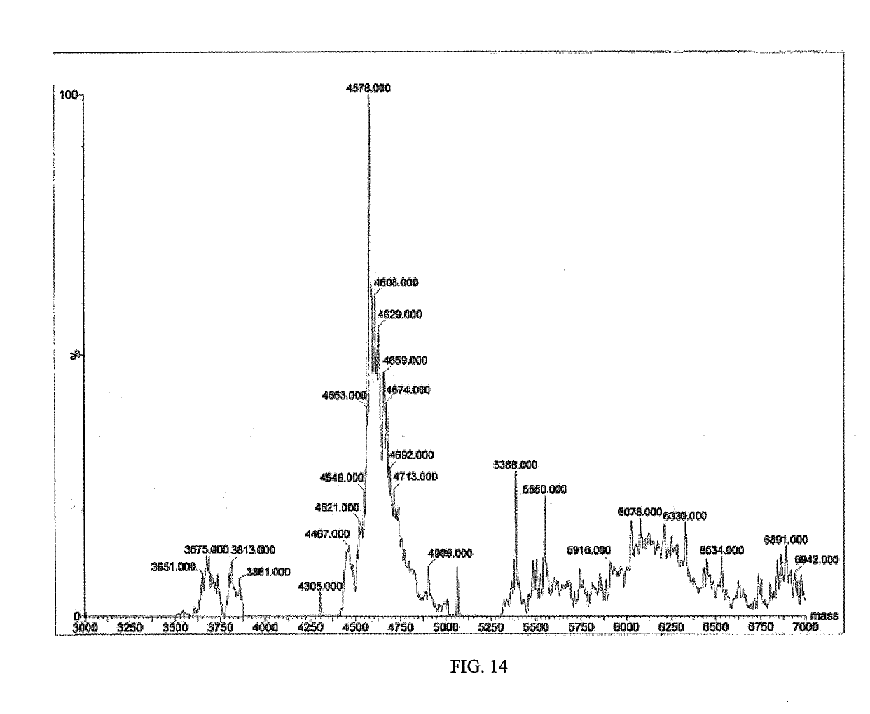

Example 6, shows the peptide hydrazide, referred to as Peptide Hydrazide (I) or Hydrazide (I), can be made from the peptide lactone. Mass Spec. data is provided in FIGS. 13 and 14.

Example 7, provides data showing the peptide hydrazide is quicker acting when the normal acid form of the peptide is made into its hydrazide form. The toxic peptide used for both compounds began with Hybrid +2. After Hybrid +2 is converted to the hydrazide the two compounds (peptide acid form and peptide hydrazide form) are different compounds but they are very similar and have the same peptide backbone. The net difference essentially is that one peptide had hydrazine was added to create the Hydrazide (I) of Hybrid +2. These two samples were then tested on flies. One of the samples, either the normal acid form of the peptide or the hydrizide form of the peptide, i.e. the Hydrazide (I), were exposed to one of two groups of flies. One group of flies was exposed to the toxic peptide in hydrazide form, i.e. Hydrazide (I), the other group of flies was exposed to the toxic peptide in its native acid form. The data provided below in Example 7 shows the hydrazide kills insects faster than the native acid form of the same peptide.

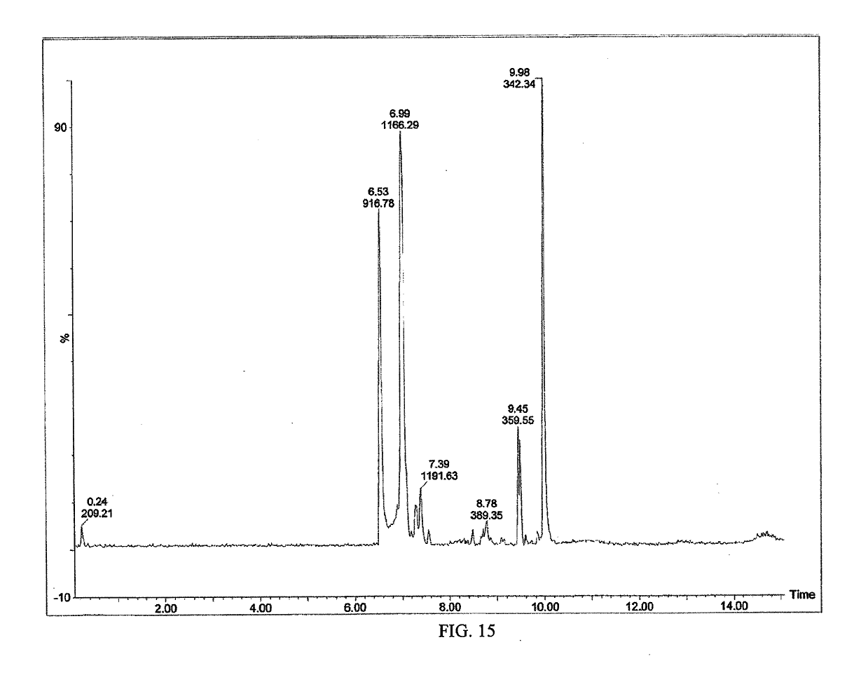

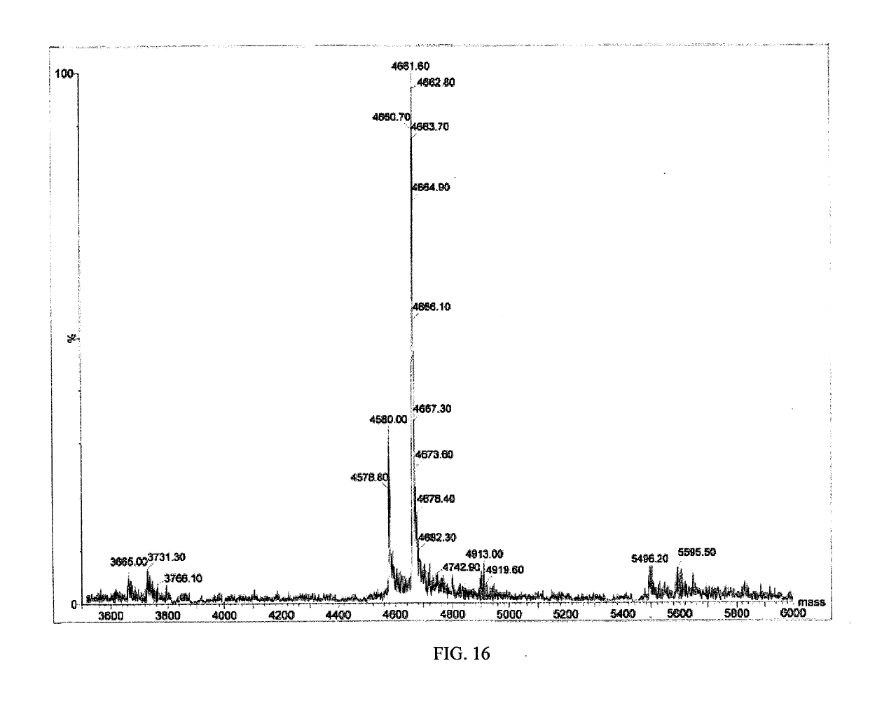

Example 8, shows how hexanal can be used to make the hydrazone form of a peptide. Example 8 starts with the hydrazide (I), hexanal is added and the result is a hydrazone, referred to here as Formula (II) or Hydrazone (II). Mass spec. data is provided in FIGS. 15 and 16.

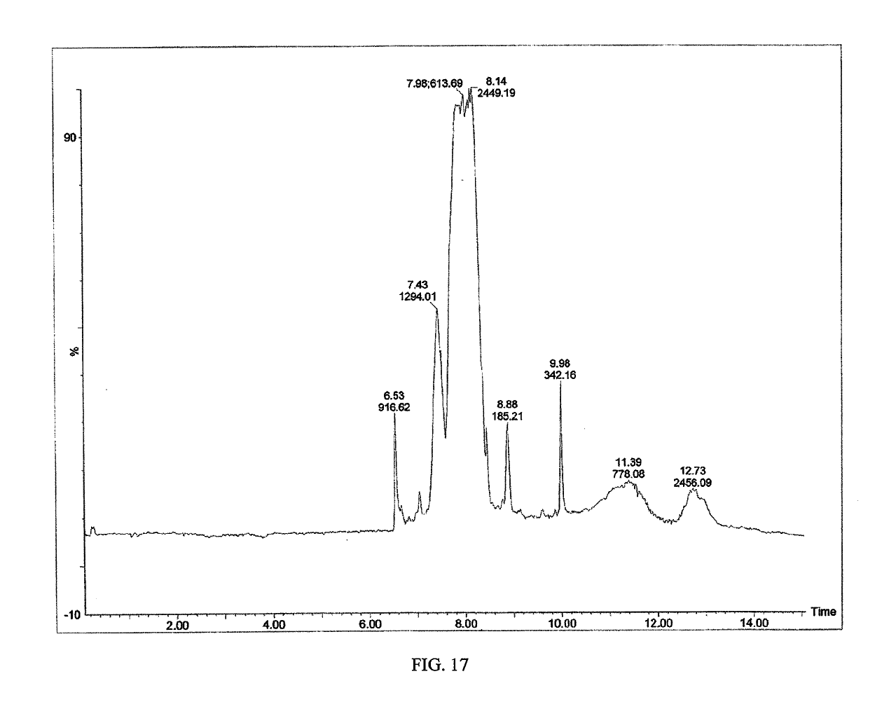

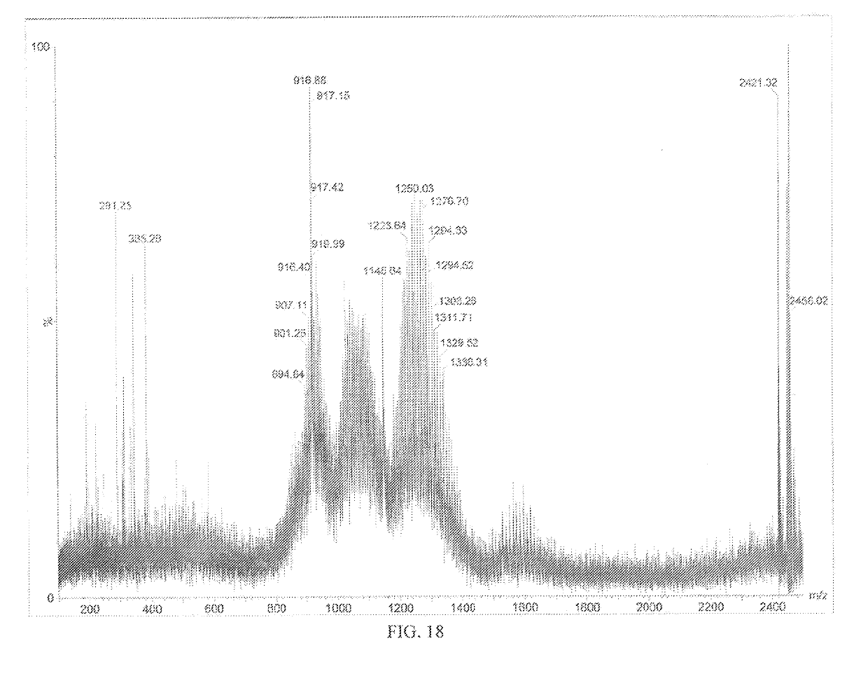

Example 9, provides for the preparation of a different hydrazone than Example 8. In Example 9 the compound “O-[2-(6-Oxocaproylamino)ethyl]-O′-methylpolyethylene glycol (IV) (MW˜2′000)” is used to make a peptide hydrazone. Mass Spec. data is provided in FIGS. 17 and 18.

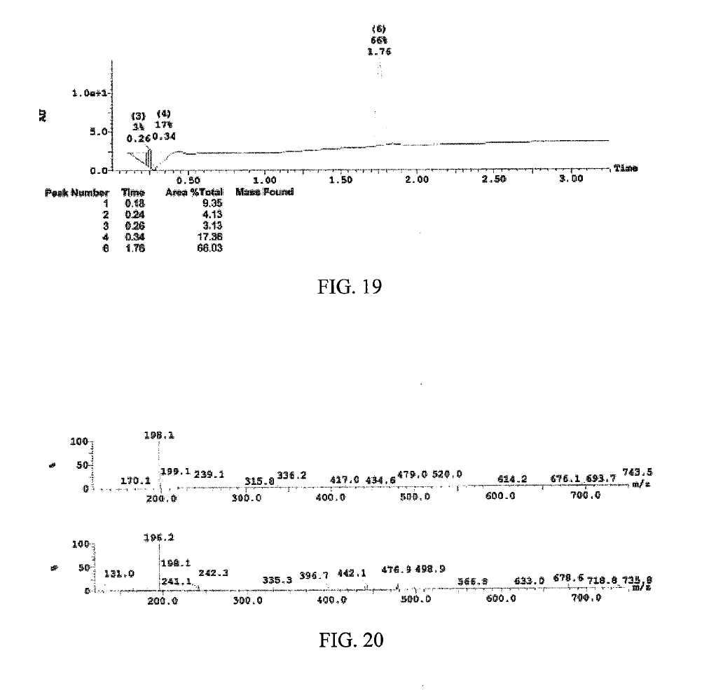

Example 10, shows another way to make a hydrazone. Here it is a hydrazone made from a hydrazide and an acrylic ketone. It is the preparation of Hydrazone (VI) from Hydrazide (I) using Acrylic Ketone (V). Mass Spec. data is provided in FIGS. 19-22.

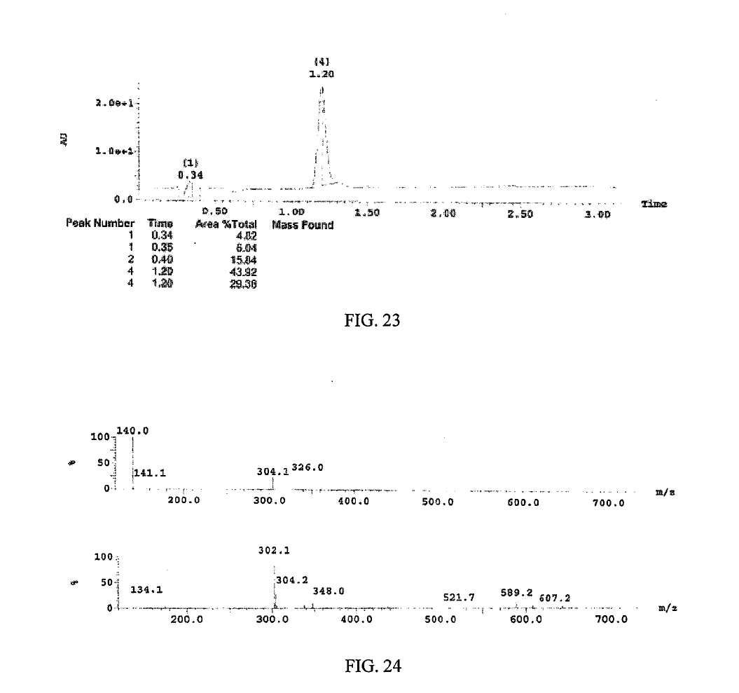

Example 11, describes the preparation of Hydrazone (IX) using a PEG4 Ketone (VIII). This example starts with Example 11(a) where 3-acetylacrylic acid and a carbodiimide are used to make PEG4 Ketone (VIII). Then, in Example 11(b), the PEG4 Ketone (VIII) and Hydrazide I are used to make Hydrazone (IX). Mass Spec. data is provided in FIGS. 23-26.

### Examples 6-11

### Details and Data

Representative formula to describe the peptide in its native acid form, the peptide lactone described in Part 1 and the peptide hydrazide of Part 2 is shown. Other hydrazides and hydrazones are described in Examples 8-11.

### Example 6

### Preparation of the Peptide Hydrazide (I)

This example shows two methods to prepare peptide hydrazide (I). In the first method, Example 6(a) the starting solution of peptide lactone is relatively pure, from an HPLC preparation. In the second method, Example 6(b), the starting solution of peptide lactone is less pure and contains both Form 1 and Form 2, that is, there is peptide mixed with the peptide lactone. Both procedures produce the same mass spec. of the peptide hydrazide.

Example 6(a). A solution of 100 mg of purified Form 2 peptide, the peptide lactone, in 1 mL of water was treated with 100 uL of hydrazine monohydrate and stirred at room temperature for 2 hours. The material was purified in portions on a prep HPLC (eluted with a gradient of acetonitrile/water/trifluoroacetic acid). Appropriate fractions were combined and concentrated under vacuum to a reduced volume. The liquid was frozen in a freezer at −80° C. and then freeze-dried on a lyopholizer to yield 36.94 mg of peptide hydrazide (I) as a white solid.

Example 6(b). A solution (25 mL) of Super Liquid Concentrate (mixture of Form 1 and Form 2 peptide, aka peptide lactone, at 14 mg/mL) was stirred overnight at 75° C. After cooling, HPLC showed mostly Form 2 peptide, the peptide lactone. The solution was treated with 2 mL of hydrazine monohydrate and stirred at room temperature for 2 hours. The material was purified in portions on a prep HPLC (eluted with a gradient of acetonitrile/water/trifluoroacetic acid). Appropriate fractions were combined and concentrated under vacuum to a reduced volume. The liquid was frozen in a freezer at −80° C. and then freeze-dried on a lyopholizer to yield 252.2 mg of peptide hydrazide (I) as a white solid. Hydrazide (I) LCMS by method B ESI/MS 4578.00 (M+H), retention time 3.6-4.1 minutes. See FIGS. 13 and 14.

### Example 7

### Fly Injection of Hydrazide (I) Compared to Hybrid+2

In Example 7 we compare a toxic peptide in its typical acid form, Form 1 or the peptide form, with the same toxic peptide after it is converted to the peptide hydrazide, or Peptide Hydrazide (I) as it is labeled in the formula provided here. The following samples are prepared for injection:
1. 100 ng/uL solution of Hydrazide (I) in water. This solution was diluted with water to make 50 ng/uL and 5 ng/uL solutions
2. 100 ng/uL solution of Hybrid+2 in water. This solution was diluted with water to make 50 ng/uL and 5 ng/uL solutions.

Prepare injected fly containers with proper labels, and punch air hole in the container lids with an 18-gauge needle. Choose flies. Use the flies on day 1 and day 2 after fully hatch-out and day 1 is the day of fully hatch-out. Turn on the CO₂ gas line and immobilize the flies in the box by input of the CO₂ gas with a needle. After flies are immobilized, transfer flies to a CO₂ plate and keep them in sleep. The flies are massed and those with mass of 12-18 mg are used for the injection bioassay.

Perform the injection. Load 100-200 μl solution into a 1 ml syringe with a 30-gauge needle and mount the loaded syringe into the microapplicator. Remove the air bubble from the syringe by turning the pushing pole of the microapplicator. Then set injection volume to 0.5 μl in the microapplicator. Inject the houseflies with 0.5 μl of the prepared solutions above from the dorsal thorax by turning the pushing pole. Inject 10 flies for each prepared solution above. Put the injected flies into the prepared cups with a lid with air holes. Add 2 Whatman #4, 4.2 cm filter paper discs. Add 1 mL of sterile nanopure water. Keep all the injected flies at room temperature. At 3 hour, 5 hour, 21 hour and 24 hour post-injection, score the injected flies. If there is more than 20% mortality in the negative control, redo the fly injection as described above. At all four scoring time points, the water and anesthesia controls had 0% mortality.

At the 5 hour time point, the 100 ng/uL concentration of hydrazide had 80% mortality while Hybrid +2 at the same concentration achieved 10% mortality.

### Example 8

### Hydrazone (II) from Hexanal

A solution of 5 mg (0.00109 mmol) of hydrazide (I) in 100 uL of water was treated with 0.16 uL (0.00133 mmol) of hexanal in 10 uL of absolute ethanol. The mixture was stirred for 1 hour. Made a stock solution of 5 mg of hexanal and 2.86 uL of acetic acid in 490 uL of absolute ethanol. Reaction was treated with 10.9 uL of the stock solution and after mixing let stand for 2 hours. The mixture was heated at −60° C. for 1 hour. LCMS by method B ESI/MS 4661.60 (M+H, hydrazone), retention time 6.8-7.1 minutes. See FIGS. 15 and 16.

### Example 9

### Hydrazone (III) from O-[2-(6-Oxocaproylamino)ethyl]-O′-methylpolyethylene glycol (IV) (MW˜2′000)

A stock solution of O-[2-(6-Oxocaproylamino)ethyl]-O′-methylpolyethylene glycol (MW˜2′000) (IV)(10.9 mg) in 100 uL of absolute ethanol was treated with 1 drop of acetic acid. Note: O-[2-(6-Oxocaproylamino)ethyl]-O′-methylpolyethylene glycol (MW˜2′000) is a mixture of compounds with a distribution around a MW of 2000 and not a single compound. A solution of 5 mg (0.00109 mmol) of hydrazide (I) in 100 uL of water was treated with 22 uL (0.0012 mmol) of the stock solution of O-[2-(6-Oxocaproylamino)ethyl]-O′-methylpolyethylene glycol (IV) (MW˜2′000). After mixing, the mixture was allowed to stand at room temperature. The remainder of the stock solution of O-[2-(6-Oxocaproylamino)ethyl]-O′-methylpolyethylene glycol (IV)(MW˜2′000) was added in portions and the mixture allowed to stand overnight after mixing. LCMS by method B ESI/MS retention time 7.2-7.6 minutes. See FIGS. 17 and 18.

### Example 10

### Hydrazone (VI) from Hydrazide (I) using Acrylic Ketone (V)

### Example 10(a)

### Preparation of Acrylic Ketone (V)

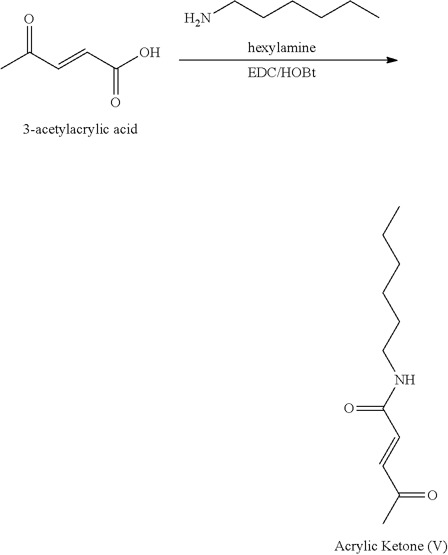

A mixture of 0.5 g (4.38 mmol) of 3-Acetylacrylic acid, 0.924 g (4.82 mmol) of N-(3-Dimethylaminopropyl)-N′-ethylcarbodiimide hydrochloride (EDC) and 0.651 g (4.82 mmol) of 1-Hydroxybenzotriazole hydrate (HOBt) in 4 mL of dichloromethane and 4 mL of tetrahydrofuran was stirred under nitrogen at room temperature for 10 minutes. The reaction was cooled in an ice bath and treated with a solution of 0.443 g (4.38 mmol) of hexylamine in 8 mL of dichloromethane. The reaction was stirred cold for 1 hour and overnight at room temperature. The reaction was diluted with dichloromethane and the organics were washed with a saturated sodium bicarbonate solution followed by a wash with water. The organic layer was dried over magnesium sulfate, filtered and concentrated under vacuum to yield a yellow solid. The solid was taken up in dichloromethane and purified on a column of silica gel (eluting with 50% ethyl acetate/hexanes). The appropriate fractions were combined and concentrated under vacuum to yield 537.06 mg of acrylic ketone (V) as a white solid. LCMS by method C ESI/MS 198.1 (M+H), 196.2 (M-H). See FIGS. 19 and 20.

### Example 10(b)

### Hydrazone (VI) from Acrylic Ketone (V)

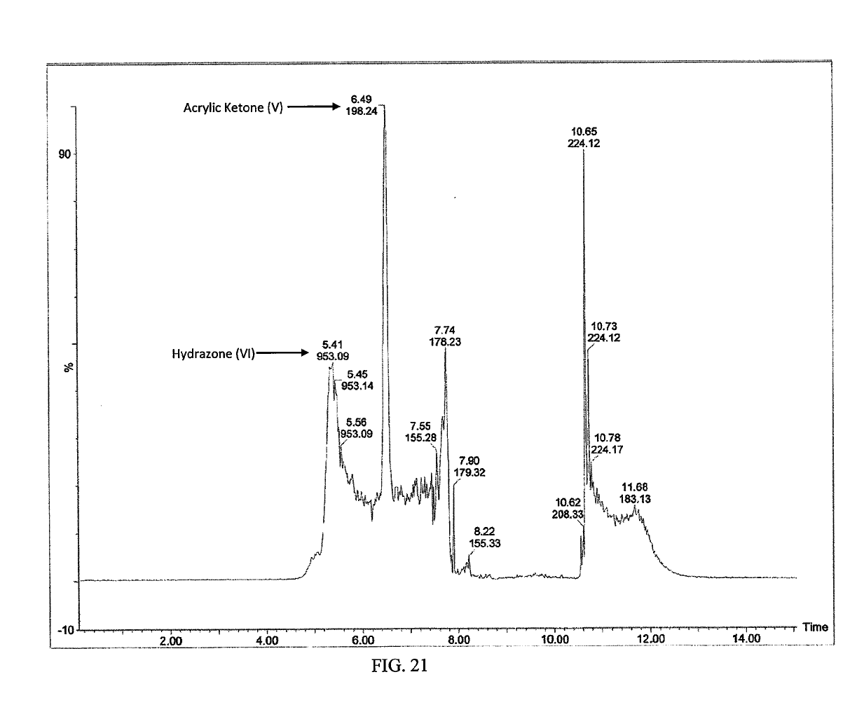

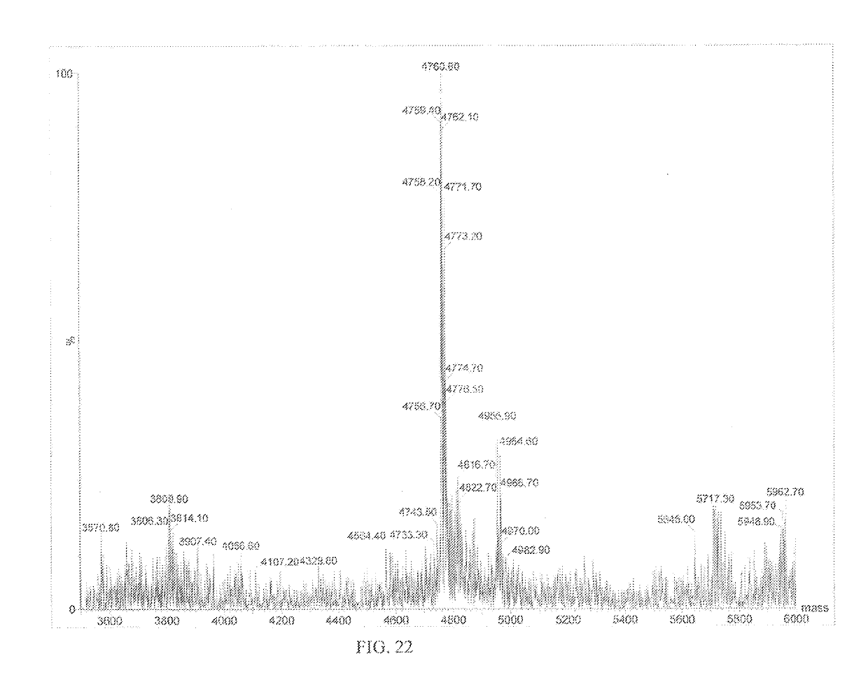

A solution of 5 mg (0.00109 mmol) of hydrazide (I) in 150 uL of water was treated in portions with 0.96 mg (0.0048 mmol) of acrylic ketone (V) in 48 uL of absolute ethanol. The mixture was stirred for ½ hour after each addition and then overnight. LCMS by method B ESI/MS 198.24 (M+H, acrylic ketone); 4760.60 (M+H, hydrazone), retention time 5.1-5.8 minutes. See FIGS. 21 and 22.

### Example 11

### Hydrazone (IX) using a PEG4 Ketone (VIII) prepared from 3-acetylacrylic acid

### Example 11(a)

### Preparation of PEG4 Ketone (VIII)

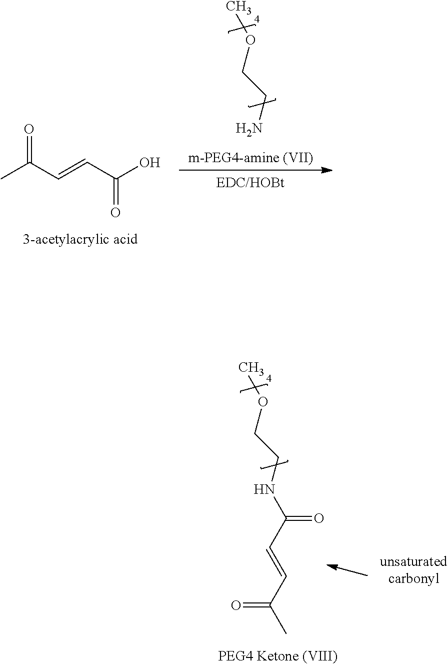

A mixture of 137.6 mg (1.21 mmol) of 3-Acetylacrylic acid, 254.3 mg (1.327 mmol) of N-(3-Dimethylaminopropyl)-N′-ethylcarbodiimide hydrochloride (EDC) and 179.25 mg (1.327 mmol) of 1-Hydroxybenzotriazole hydrate (HOBt) in 1 mL of dichloromethane and 1 mL of tetrahydrofuran was stirred under nitrogen at room temperature for 10 minutes. The reaction was cooled in an ice bath and treated with a solution of 250 mg (1.21 mmol) of m-PEG4-amine (VII) in 2 mL of dichloromethane. The reaction was stirred cold for 1 hour and overnight at room temperature. The reaction was diluted with dichloromethane and the organics were washed with a saturated sodium bicarbonate solution followed by a wash with water. The organic layer was dried over magnesium sulfate, filtered and concentrated under vacuum to yield 302.29 mg of PEG4 Ketone (VIII) as an oil. LCMS by method C ESI/MS 304.1 (M+H), 302.1 (M−H). See FIGS. 23 and 24.

### Example 11(b)

### Hydrazone (IX) using PEG4 Ketone (VIII)

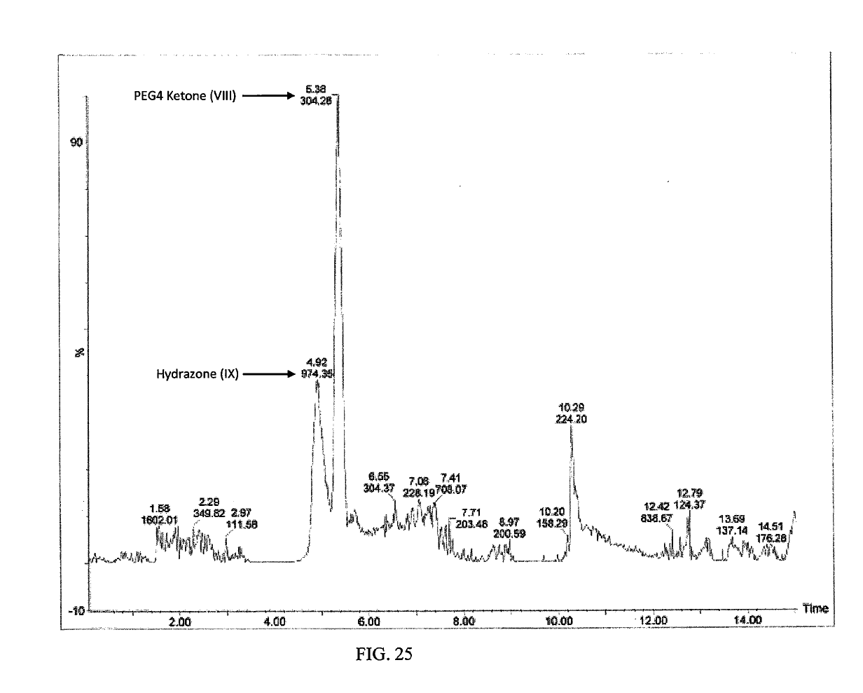

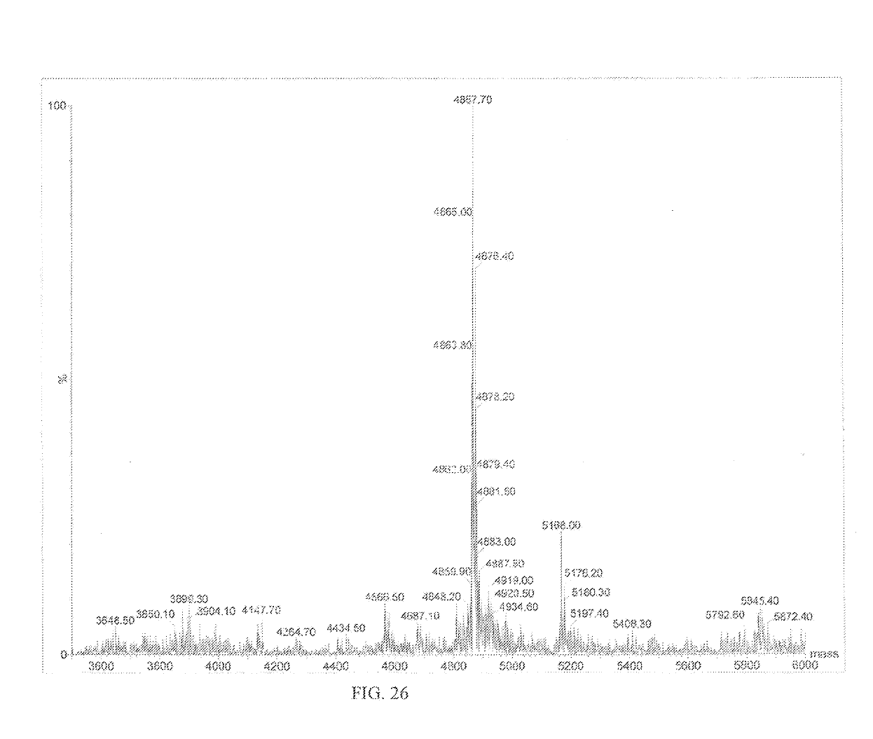

A solution of 5 mg (0.00109 mmol) of hydrazide (I) in 150 uL of water was treated in portions with 2.0 mg (0.0066 mmol) of PEG4 Ketone (VIII). The mixture was stirred for ½ hour after each addition. LCMS by method B ESI/MS 304.28 (M+H, PEG4 ketone); 4867.70 (M+H, hydrazone) retention time 4.7-5.1 minutes. See FIGS. 25 and 26.

The examples are intended to illustrate and not limit the claims and the claimed invention. One ordinarily skilled in the art would be expected to be able to make numerous variations and different version of what is shown here.

1-23. (canceled)

24. A process of increasing the activity or toxicity of a peptide, including a toxic peptide, comprising the following steps:
a) mix said peptide with water to make an aqueous solution or aqueous emulsion of said peptide in a liquid or semi-liquid form, wherein the aqueous solution or aqueous emulsion is comprised of at least 10% water,
b) measure the pH of said peptide in the aqueous solution or aqueous emulsion,
c) adjust the pH of said solution or emulsion to a pH of between about 1.0 and about 6.5; between about 2.0 and about 6.0; less than about 7.0; less than 7.0; between about 2.5 and about 5.5; between about 3.0 and about 5.0; between about 3.0 and about 4.0; or: adjusted to a pH of about 3.2, 3.3, 3.4, 3.5, 3.6, 3.7 or 3.8

25. The process of claim 24 wherein said pH adjustment is made using a strong or weak acid;
wherein, a strong acid is selected from any of the following acids or combinations of acids: chloric acid (HClO₃), hydrochloric acid (HCl), hydrobromic acid (HBr), hydroiodic acid (HI), phosphoric acid (H₃PO₄), sulfuric acid (H₂SO₄). Perchloric acid (HClO₄), and Nitric acid (HNO₃), either individually or in combination,
wherein preferred strong acids are selected from phosphoric, sulfuric or nitric acid;
wherein weak acids selected from acetic acid and/or oxalic acid, either individually or in combination,
and optionally,
wherein during the pH adjustment, the aqueous solution or aqueous emulsion is exposed to heat, including a dry heat, wherein optionally a dry heat is a temperature increase without steam or pressure; or with pressure and or steam; or any combination thereof and where optionally after said pH adjustment the peptide is dried to a dry powder or granular form,
and optionally,
removing any one or more covalently bound 2H+O or molecules from a peptide while said peptide is in an aqueous solution or emulsion by the reduction of the pH of the solution or emulsion to less than 7.0, and optionally,
wherein said peptide is any peptide, any toxic peptide, any peptide in the sequence listing; the peptide that is SEQ ID NO. 119; the peptide that is SEQ ID NO. 121, and any peptide described in the specification and claims,
and optionally,
wherein said insecticidal composition of the peptides of toxic peptides are incorporated into a formulation suitable for application to the locus of an insect to be treated with the peptide or toxic peptide,
and optionally,
wherein when one or more covalently bound 2H+O or molecules are removed the pH of the peptides in aqueous solution or emulsion is reduced to less than 7.0.

26. A process of claim 25 wherein said toxicity and/or activity of a peptide is increased, comprising the following steps:
a) prepare said peptide as a pure Form 1 or peptide acid and or composition containing less than about 10% water,
b) place said Form 1 peptide or peptide acid in a controllable chamber or heating platform;
c) heat said peptide to a desired temperature, with or without pressure, with or without steam;
d) maintain the heated peptide at the desired temperature, pressure and steam until the desired amount of Form 1 peptide or peptide acid Converts to Form 2 peptide or peptide lactone;
optionally and independently; wherein steps a) to d) are performed in the following conditions:
optionally and independently; wherein the controllable chamber can maintain temperatures from 0 to 500° C. and pressures from atmospheric to 500 psi;
optionally and independently; wherein the peptide is heated to about the following temperatures; heated to at least about 10° C. but to no more than a maximum temperature selected from about 200° C., 300° C., 400° C. or 500° C.;
optionally and independently; wherein the peptide is heated to at least from a temperature selected from about any of the following temperatures, temperature ranges or combinations of ranges of temperatures: 10° C. to 20° C.; 20° C. to 30° C.; 30° C. to 40° C.; 40° C. to 50° C.; 50° C. to 60° C.; 60° C. to 70° C.; 70° C. to 80° C.; 80° C. to 90° C.; 90° C. to 100° C.; 100° C. to 110° C., 110° C. to 120° C., 120° C. to 130° C., 130° C. to 140° C., 140° C. to 150° C., 150° C. to 160° C., 160° C. to 170° C., 170° C. to 180° C., 180° C. to 190° C., 190° C.-200° C., 200° C. to 210° C., 210° C. to 220° C., 220° C. to 230° C., 230° C. to 240° C., 240° C. to 250° C., 250° C. to 260° C., 260° C. to 270° C., 270° C. to 280° C., 280° C. to 290° C., 290° C. to 300° C., 300° C. to 400° C. and 400° C. to 500° C.;
optionally and independently; wherein the pressure is selected from any of the following pressures or ranges of pressures: a) from about 10 psi to about 40 psi, b) from about 15 psi to about 35 psi, c) from about 18 psi to about 25 psi, d) about 21 psi;
optionally and independently; wherein the peptides are maintained at the chosen temperature and pressure range from the following periods depending on the temperature and pressure chosen: a) from about 5 minutes to about 40 minutes; b) from about 10 minutes to about 30 minutes; c) from about 15 minutes to about 25 minutes; d) about 21 minutes;
optionally and independently; wherein the peptide is heated to and maintained at the following temperatures and pressures and times: a) between from about 100° C. to about 140° C.; at a pressure of from about 10 psi to about 40 psi; for from about 5 minutes to about 40 minutes; b) between from about 110° C. to about 130° C.; at a pressure of from about 15 psi to about 35 psi; for from about 10 minutes to about 30 minutes; c) between from about 115° C. to about 125° C.; at a pressure of from about 18 psi to about 25 psi; for from about 15 minutes to about 25 minutes; d) of about 121° C., at a pressure of about 21 psi, for about 20 minutes;
optionally and independently; wherein the pressure is no greater than atmospheric pressure and the temperature is selected from the temperatures of at least 50° C. to 60° C. or greater;
optionally and independently; wherein the following temperatures, temperature ranges or combinations of ranges of temperatures are used: 50° C. to 60° C.; 60° C. to 70° C.; 70° C. to 80° C.; 80° C. to 90° C.; 90° C. to 100° C.; 100° C. to 110° C., 110° C. to 120° C., 120° C. to 130° C., 130° C. to 140° C., 140° C. to 150° C., 150° C. to 160° C., 160° C. to 170° C., 170° C. to 180° C., 180° C. to 190° C., 190° C.-200° C., 200° C. to 210° C., 210° C. to 220° C., 220° C. to 230° C., 230° C. to 240° C., 240° C. to 250° C., 250° C. to 260° C., 260° C. to 270° C., 270° C. to 280° C., 280° C. to 290° C., 290° C. to 300° C., 300° C. to 400° C. and 400° C. to 500° C.

27. The process of claim 26 wherein said peptide is treated according to any of the multistep procedures provided below, including wherein said peptide is:
a) heated and maintained at a temperature of more than about 100° C. for at least about 1 hr.;
b) heated and maintained at a temperature of between about from 80° C. to about 120° C. for at least about 2 hr.;
c) heated and maintained at a temperature of between about from 50° C. to about 80° C. for at least about 3 hr;
alternatively and optionally wherein said peptide is treated according to any of the multistep procedures provided below, including wherein said peptide is
a) heated and maintained at a temperature of more than about 180° C., and a pressure of at least about 5 psi for at least about 5 minutes;
b) heated and maintained at a temperature of more than about 100° C., and a pressure of at least about 10 psi for at least about 10 minutes;
c) heated and maintained at a temperature of between about from 80° C. to about 120° C., and a pressure of at least about 10 psi, for at least about 30 minutes.; or
d) heated and maintained at a temperature of between about from 50° C. to about 80° C. for at least about 1 hr.;
alternatively and optionally wherein said peptide is treated according to any of the multistep procedures provided below, including wherein said peptide is
a) heated and maintained at a temperature of between about 200° C. to about 300° C., and a pressure of between about 5 to about 10 psi for between about 5 to about 10 minutes;
b) heated and maintained at a temperature of between about 150° C., and about 200° C., and a pressure of between about 10 to about 30 psi for between about 5 to about 30 minutes;
c) heated and maintained at a temperature of between about from 80° C. to and about 150° C., and a pressure of between about 10 to about 20 psi for between about 20 to about 60 minutes; or
d) heated and maintained at a temperature of between about from 50° C. to about 80° C. and a pressure of between about 10 to about 40 psi for between about 30 to about 60 minutes. alternatively wherein said peptide is
a) heated and maintained at a temperature of between about 110° C., and about 130° C., and a pressure of between about 10 to about 20 psi for between about 10 to about 20 minutes; or
b) heated and maintained at a temperature of about 121° C., and a pressure about 21 psi for about 20 minutes.

28. A toxic peptide treated to have increased activity or toxicity according to the following steps:
a) mix said peptide with water to make an aqueous solution or aqueous emulsion of said peptide in a liquid or semi-liquid form, wherein the aqueous solution or aqueous emulsion is comprised of at least 10% water,
b) measure the pH of said peptide in the aqueous solution or aqueous emulsion,
c) adjust the pH of said solution or emulsion to a pH of between about 1.0 and about 6.5; between about 2.0 and about 6.0; less than about 7.0; less than 7.0; between about 2.5 and about 5.5; between about 3.0 and about 5.0; between about 3.0 and about 4.0; or: adjusted to a pH of about 3.2, 3.4, 3.5., 3.6, or 3.8.

29. The toxic peptide of claim 28 wherein said pH adjustment is made using a strong or weak acid; wherein if using a strong acid the strong acid pH adjustment is selected from any of the following acids, or combinations of acids: chloric acid (HClO₃), hydrochloric acid (HCl), hydrobromic acid (HBr), hydroiodic acid (HI), phosphoric acid (H₃PO₄), sulfuric acid (H₂SO₄). Perchloric acid (HClO₄), and Nitric acid (HNO₃), with attention to using a strong acid selected from phosphoric, sulfuric or nitric acid: wherein if using a weak acid the weak acid pH adjustment is made using a weak acid selected from acetic acid and/or oxalic acid, either individually or in combination,
and optionally,
where, during the pH adjustment, the aqueous solution or aqueous emulsion is exposed to heat, including a dry heat i.e. a temperature increase without steam or pressure; or with pressure and or steam; or any combination thereof and where optionally after said pH adjustment the peptide is dried to a dry powder or granular form.
and optionally,
removing any one or more covalently bound 2H+O or molecules from a peptide while said peptide is in an aqueous solution or emulsion by the reduction of the pH of the solution or emulsion to less than 7.0,
and optionally,
wherein said peptide, is any toxic peptide, any peptide in the sequence listing; the peptide that is SEQ ID NO. 119; the peptide that is SEQ ID NO. 121, any peptide described in the specification and claims,
and optionally,
wherein said toxic peptides are incorporated into a formulation suitable for application to the locus of an insect to be treated with the peptide or toxic peptide, and optionally,
wherein when any one or more covalently bound 2H+O or molecules are removed the pH of the peptide in aqueous solution or emulsion is reduced to less than 7.0.

30. A toxic peptide of claim 29, treated to have more toxicity or and/or activity according to the following steps:
a) prepare said peptide as a pure Form 1 or peptide acid or composition containing less than about 10% water,
b) place said Form 1 peptide or peptide acid in a controllable chamber or heating platform;
c) heat said peptide to a desired temperature, with or without pressure, with or without steam;
d) maintain the heated peptide at the desired temperature, pressure and steam until the desired amount of Form 1 peptide or peptide acid Converts to Form 2 peptide or peptide lactone;
and optionally and independently; wherein steps a) to d) are performed in the following conditions:
optionally and independently; wherein the controllable chamber can maintain temperatures from 0 to 500° C. and pressures from atmospheric to 500 psi;
optionally and independently; wherein the peptide is heated to about the following temperatures; heated to at least about 10° C. but to no more than a maximum temperature selected from about 200° C., 300° C., or at most 400° C.;
optionally and independently; wherein the peptide is heated to at least from a temperature selected from about any of the following temperatures, temperature ranges or combinations of ranges of temperatures: 10° C. to 20° C.; 20° C. to 30° C.; 30° C. to 40° C.; 40° C. to 50° C.; 50° C. to 60° C.;
60° C. to 70° C.; 70° C. to 80° C.; 80° C. to 90° C.; 90° C. to 100° C.; 100° C. to 110° C., 110° C. to 120° C., 120° C. to 130° C., 130° C. to 140° C., 140° C. to 150° C., 150° C. to 160° C., 160° C. to 170° C., 170° C. to 180° C., 180° C. to 190° C., 190° C. - 200° C., 200° C. to 210° C., 210° C. to 220° C., 220° C. to 230° C., 230° C. to 240° C., 240° C. to 250° C., 250° C. to 260° C., 260° C. to 270° C., 270° C. to 280° C., 280° C. to 290° C., 290° C. to 300° C., 300° C. to 400° C. and 400° C. to 500° C.;
optionally and independently; wherein the pressure is selected from any of the following pressures or ranges of pressures: a) from about 10 psi to about 40 psi, b) from about 15 psi to about 35 psi, c) from about 18 psi to about 25 psi, d) about 21 psi;
optionally and independently; wherein the peptides are maintained at the chosen temperature and pressure range from the following periods depending on the temperature and pressure chosen: a) from about 5 minutes to about 40 minutes; b) from about 10 minutes to about 30 minutes; c) from about 15 minutes to about 25 minutes; d) about 21 minutes;
optionally and independently; wherein the peptide is heated to and maintained at the following temperatures and pressures and times: a) between from about 100° C. to about 140° C.; at a pressure of from about 10 psi to about 40 psi; for from about 5 minutes to about 40 minutes; b) between from about 110° C. to about 130° C.; at a pressure of from about 15 psi to about 35 psi; for from about 10 minutes to about 30 minutes; c) between from about 115° C. to about 125° C.; at a pressure of from about 18 psi to about 25 psi; for from about 15 minutes to about 25 minutes; d) of about 121° C., at a pressure of about 21 psi, for about 20 minutes;
optionally and independently; wherein the pressure is no greater than atmospheric pressure and the temperature is selected from the temperatures of at least 50° C. to 60° C. or greater;
optionally and independently; wherein the following temperatures, temperature ranges or combinations of ranges of temperatures are used: 50° C. to 60° C.; 60° C. to 70° C.; 70° C. to 80° C.; 80° C. to 90° C.; 90° C. to 100° C.; 100° C. to 110° C., 110° C. to 120° C., 120° C. to 130° C., 130° C. to 140° C., 140° C. to 150° C., 150° C. to 160° C., 160° C. to 170° C., 170° C. to 180° C., 180° C. to 190° C., 190° C.-200° C., 200° C. to 210° C., 210° C. to 220° C., 220° C. to 230° C., 230° C. to 240° C., 240° C. to 250° C., 250° C. to 260° C., 260° C. to 270° C., 270° C. to 280° C., 280° C. to 290° C., 290° C. to 300° C., 300° C. to 400° C. and 400° C. to 500° C.

31. The peptide of claim 29 wherein said peptide is treated according to any of the multistep procedures provided below including wherein said peptide is:
a) heated and maintained at a temperature of more than about 100° C. for at least about 1 hr.;
b) heated and maintained at a temperature of between about from 80° C. to about 120° C. for at least about 2 hr.;
c) heated and maintained at a temperature of between about from 50° C. to about 80° C. for at least about 3 hr;
alternatively and optionally wherein said peptide is treated according to any of the multistep procedures provided below including wherein said peptide is:
a) heated and maintained at a temperature of more than about 180° C., and a pressure of at least about 5 psi for at least about 5 minutes;
b) heated and maintained at a temperature of more than about 100° C., and a pressure of at least about 10 psi for at least about 10 minutes;
c) heated and maintained at a temperature of between about from 80° C. to about 120° C., and a pressure of at least about 10 psi, for at least about 30 minutes.; or
d) heated and maintained at a temperature of between about from 50° C. to about 80° C. for at least about 1 hr.;
alternatively and optionally wherein said peptide is treated according to any of the multistep procedures provided below including wherein said peptide is:
a) heated and maintained at a temperature of between about 200° C. to about 300° C., and a pressure of between about 5 to about 10 psi for between about 5 to about 10 minutes;
b) heated and maintained at a temperature of between about 150° C., and about 200 ° C., and a pressure of between about 10 to about 30 psi for between about 5 to about 30 minutes;
c) heated and maintained at a temperature of between about from 80° C. to and about 150° C., and a pressure of between about 10 to about 20 psi for between about 20 to about 60 minutes; or
d) heated and maintained at a temperature of between about from 50° C. to about 80° C. and a pressure of between about 10 to about 40 psi for between about 30 to about 60 minutes.
alternatively and optionally wherein said peptide is treated according to any of the multistep procedures provided below including wherein said peptide is:
a) heated and maintained at a temperature of between about 110° C., and about 130° C., and a pressure of between about 10 to about 20 psi for between about 10 to about 20 minutes; or
b) heated and maintained at a temperature of about 121° C., and a pressure about 21 psi for about 20 minutes.

32. A process of converting a toxic peptide or an insect predator peptide from the peptide lactone form to the peptide hydrazide form comprising:
mixing a toxic peptide or an insect predator peptide lactone with hydrazine and purifying to obtain the peptide hydrazide
and optionally
converting a peptide in a hydrazide form into a peptide hydrazone form comprising acidifying complex glycols with a strong or weak acid, and
adding a peptide hydrazide and mixing well to make peptide hydrazone.

33. The process of claim 32, wherein the peptide lactone is prepared in water, hydrazine monohydrate is added and the mixture is stirred to form the peptide hydrazide which is optionally frozen, thawed and purified to obtain purified peptide hydrazide.

34. The process of claim 33,wherein the a toxic peptide or an insect predator peptide varies in size from about 20 amino acids to about 50 amino acids and has 2, 3 or 4 cystine bonds, or alternatively having 3 or 4 cystine bonds; and optionally using any of the alternatives below:
A) wherein the peptide lactone is prepared from any peptide in the sequence listing and any peptide in the sequence listing or any peptide with more than 80% homology to any peptide in the sequence listing, or any sequence having more than 85%, 90%, 95% or 99% homology and 3 or 4 cystine bonds; and
alternatively and optionally wherein said peptide is treated according to any of the multistep procedures provided below including wherein the peptide being converted is named the Hybrid+2 peptide wherein either method 1 or method 2 below can be used;
method 1), comprising;
a) start with a solution of 100 mg of purified Form 2 peptide, the Hybrid+2 peptide lactone, in 1 mL of water,
b) treat the 1 mL of 100 mg peptide lactone with 100 uL of hydrazine monohydrate and stir at room temperature to form the peptide hydrazide, optionally for 2 hours,
c) purify the solution of peptide hydrazide on a prep HPLC (eluted with a gradient of acetonitrile/water/trifluoroacetic acid),
d) select appropriate fractions of peptide hydrazide,
e) combine appropriate fractions of peptide hydrazide and concentrating under vacuum to reduce the volume,
f) freeze the reduced volume of peptide hydrazide, at below zero temperature, optionally at −80° C.,
g) freeze-dry the Hybrid+2 peptide hydrazide, optionally on a lyopholizer, to obtain Hybrid +2 peptide hydrazide (I);
or
method 2), comprising:
a) stir a solution of 25 mL of Super Liquid Concentrate, which is a mixture of Form 1, the peptide acid and Form 2, optionally at about 50° C. to 90° C., optionally at 75° C.,
b) let the solution cool,
c) treat solution with hydrazine monohydrate, optionally 2 mL, and stir, optionally at room temperature for 2 hours,
d) purify portions on a prep HPLC, optionally eluted with a gradient of acetonitrile/water/trifluoroacetic acid),
e) combine and concentrate fractions, reduce volume, optionally under vacuum,
f) freeze remaining liquid, optionally freeze at −80° C. and lyopholize to produce Hybrid +2 peptide hydrazide.

35. The process of claim 32 of making a peptide hydrazone by the process of converting an insect predator peptide from the peptide hydrazide to the peptide hydrazone comprising:
a) mix a solution of hydrazide in water and add hexanal in ethanol, stir,
b) treat with a stock solution made of hexanal, acetic acid and ethanol, stir,
c) add a stock solution made from hexanal, acetic acid and ethanol,
d) mix, let stand and then optionally heat to produce the hydrazone.

36. The process of claim 35 wherein the product is the peptide Hydrazone (II), comprising,
a) a solution of hydrazide (I) in water with hexanal in ethanol, stired,
b) with some added some stock solution of claim 35,
d) mixed and allowed to stand, with optional heating, to produce Hydrazone (II) (II).

37. The process of claim 32 of making a peptide hydrazide made by the process of converting an insect predator peptide from the peptide hydrazide to the peptide hydrazone comprising, acidifying complex glycols with a strong or weak acid, and adding a peptide hydrazide and mixing well to make peptide hydrazone.

38. The process of claim 37 wherein the peptide hydrazide converted to the peptide Hydrazone (III), comprising:
a) adding 1 drop of acetic acid to a stock solution of the mixture of compounds refered to as O-[2-(6-Oxocaproylamino)ethyl]-O′-methylpolyethylene glycol (IV) in ethanol,
b) use the stock solution of O-[2-(6-Oxocaproylamino)ethyl]-O′-methylpolyethylene glycol (IV) (MW˜2′000) treated with acetic acid from step a and add it to a solution of hydrazide (I) in water,
c) mix and allow to stand at room temperature,
d) add the remainder of the stock solution of O-[2-(6-Oxocaproylamino)ethyl]-O′-methylpolyethylene glycol (IV)(MW˜2′000) in portions and allow the mixture to stand overnight after mixing to produce Peptide Hydrazone (III).

39. The process of claim 37 of making a peptide hydrazone made by the process of converting an insect predator peptide from the peptide hydrazide to the peptide hydrazone comprising, adding an acrylic ketone to a hydrazide to make a hydrazone.

40. The process of claim 39, wherein the product is the peptide Hydrazone (VI) comprising, adding acrylic ketone (V) in ethanol to a solution of hydrazide (I) in water and mixing.

41. The process of claim 39, wherein the product is the peptide Hydrazone (IX) comprising adding PEG4 Ketone (VIII) to a solution of hydrazide (I) in water, and mixing to make Hydrazone (IX).

42. A process of preparing a peptide and or the peptide produced by the process and or an insecticidal composition produced by the process described as removing any one or more covalently bound 2H+O, or H₂O or molecules from a peptide; including any toxic peptide with any one or more covalently bound 2H+O or molecules removed under any of the conditions, temperatures, pressures and pH or acidic conditions, either alone or in combination as described herein or found in the specification or claims including any of the peptides, hydrazides or hydrazones produced from any of the procedures described in the specification and claims or use of any of these peptides as insecticidal compositions of the peptides produced by any of the processes described in the specification and claims and then used in a formulation suitable for application to the locus of an insect.

43. An insect predator peptide hydrazide, made by the process of converting an insect predator peptide from the peptide lactone form to the peptide hydrazide form, comprising:
mixing an insect predator peptide lactone with hydrazine and purifying the mixture to obtain the peptide hydrazide,
and or
an insect peptide hydrazone made from an insect predator peptide hydrazide that is converted to the peptide hydrazone.

44. The insect predator peptide hydrazide of claim 43, wherein the peptide lactone is prepared in water, hydrazine monohydrate is added and the mixture is stirred to form the peptide hydrazide which is optionally frozen, thawed and purified to obtain purified peptide hydrazide.

45. The insect predator peptide hydrazide of claim 44,
wherein the insect predator peptide is selected from any of, or a combination of, the following:
A) an insect predator peptide from about 20 amino acids to about 50 amino acids, with either 2, 3 or 4 cystine bonds, or alternatively having only 3 or 4 cystine bonds, or alternatively having only 4 cystine bonds,
Or
B) an insect predator peptide wherein the peptide lactone form is prepared from any peptide in the sequence listing and any peptide in the sequence listing or any peptide with more than 80% homology to any peptide in the sequence listing, or any sequence having more than 85%, 90%, 95% or 99% homology and 3 or 4 cystine bonds.

46. The insect predator peptide hydrazide of claim 45 that is made from the insect predator peptide named the “Hybrid +2” peptide, wherein either method 1 or method 2, below, can be used to make the insect predator peptide hydrazide, comprising either method 1 or method 2, below;
method 1, comprising:
a) start with a solution of 100 mg of purified Form 2 peptide, the Hybrid+2 peptide lactone, in 1 mL of water,
b) treat the 1 mL of 100 mg peptide lactone with 100 uL of hydrazine monohydrate and stir at room temperature to form the peptide hydrazide, optionally for 2 hours,
c) purify the solution of peptide hydrazide on a prep HPLC (eluted with a gradient of acetonitrile/water/trifluoroacetic acid),
d) select appropriate fractions of peptide hydrazide,
e) combine appropriate fractions of peptide hydrazide and concentrating under vacuum to reduce the volume,
f) freeze the reduced volume of peptide hydrazide, at below zero temperature, optionally at −80° C.,
g) freeze-dry the Hybrid +2 peptide hydrazide, optionally on a lyopholizer , to obtain Hybrid +2 peptide hydrazide (I);
or
method 2, comprising:
a) stir a solution of 25 mL of Super Liquid Concentrate, which is a mixture of Form 1, the peptide acid and Form 2, optionally at about 50° C. to 90° C., optionally at 75° C.,
b) let the solution cool,
c) treat solution with hydrazine monohydrate, optionally 2mL, and stir, optionally at room temperature for 2 hours,
d) purify portions on a prep HPLC, optionally eluted with a gradient of acetonitrile/water/trifluoroacetic acid),
e) combine and concentrate fractions, reduce volume, optionally under vacuum,
f) freeze remaining liquid, optionally freeze at −80° C. and lyopholize to produce Hybrid +2 peptide hydrazide.

47. A insect peptide according to claim 43 wherein the peptide is a peptide hydrazone made from an insect predator peptide hydrazide that is converted to the peptide hydrazone comprising,
a) mixing a solution of hydrazide in water and adding hexanal in ethanol to the solution of hydrazide, mix, then,
b) treating or add to the stirred mixture a stock solution of hexanal, acetic acid and ethanol, stiring, mix, let stand and then,
d) optionally, heat to produce the hydrazone.

48. The insect peptide hydrazone of claim 47, that is the peptide Hydrazone (II) comprising the process of
a) mixing a solution hydrazide (I) in water with hexanal in ethanol, stir,
b) adding some stock solution of hexanal, acetic acid and ethanol,
d) mixing and let stand then
e) optionally heating to produce Hydrazone (II).

49. The insect peptide hydrazone of claim 48 that is Hydrazone (III) made according to the following steps;
a) adding 1 drop of acetic acid to a stock solution of the mixture of compounds refered to as O-[2-(6-Oxocaproylamino)ethyl]-O′-methylpolyethylene glycol (IV) in ethanol, to make a stock solution,
b) use the stock solution of O-[2-(6-Oxocaproylamino)ethyl]-O′-methylpolyethylene glycol (IV) (MW˜2′000) treated with acetic acid from step a and add it to a solution of hydrazide (I) in water,
c) mix and allow to stand at room temperature,
d) add the remainder of the stock solution of O-[2-(6-Oxocaproylamino)ethyl]-O′-methylpolyethylene glycol (IV)(MW˜2′000) in portions and allow the mixture to stand overnight after mixing to produce peptide Hydrazone (III).

50. A peptide hydrazone of claim 47, made by converting an insect peptide to and insect peptide hydrazide and from the peptide hydrazide to peptide hydrazone is made according to the following process; comprising: adding an acrylic ketone to a peptide hydrazide to make a peptide hydrazone; where the peptide hydrazone is selected from either of the hydrazones made according to the following procedures:
method 1, wherein the hydrazone is Hydrazone (VI) made from adding acrylic ketone (V) in ethanol to a solution of hydrazide (I) in water and mixing and/or
method 2 wherein the hydrazone is Hydrazone (IX) made from adding PEG4 Ketone (VIII) to a solution of hydrazide (I) in water, and mixing to make Hydrazone (IX).
